(19) United States
(12) Patent Application Publication
(10) Pub. No.: US 2009/0123536 A1
(43) Pub. Date: May 14, 2009

(54) Oral Pharmaceutical Form of Losartan

(75) Inventors: Catherine Castan, Orlienas (FR); Florence Guimberteau, Montussan (FR); Remi Meyrueix, Lyon (FR); Gerard Soula, Meyzieu (FR)

Correspondence Address: PATTON BOGGS LLP, 8484 WESTPARK DRIVE, SUITE 900, MCLEAN, VA, 22102, US

(73) Assignee: Flamel Technologies, S.A., Venissieux Cedex (FR)

(21) Appl. No.: 11/884,534

(22) PCT Filed: Feb. 21, 2006
(86) PCT No.: PCT/EP2006/060158

§ 371 Date: May 15, 2008

(30) Foreign Application Priority Data
Feb. 21, 2005 (FR) 0550476
Apr. 6, 2005 (FR) 0503451

(51) Int. Cl. A61K 9/54; A61K 31/4178; A61K 9/16; A61K 9/26

(52) U.S. Cl. 424/458; 514/381; 424/484; 424/490; 424/469

(57) ABSTRACT

The field of the present invention is that of oral pharmaceutical forms of losartan, and also treatments and administration methods relating thereto.

The invention relates to the use, in an oral pharmaceutical form comprising losartan, of a coating or matrix including said losartan and allowing controlled release of said losartan, such that this form orally administered to a sample of individuals leads, irrespective of the fed or fasted state of the individuals, to a reduction of the interindividual standard deviation of the Cmax, which ensures lower variability of the efficacy and of the therapeutic safety of the pharmaceutical form relative to an immediate-release pharmaceutical form of losartan administered to this same sample of individuals, at the same dose.

Another aim of the invention is to provide an oral pharmaceutical form of losartan that can be administered once a day and that is just as effective as the “one dose intake per day” forms and the “two dose intakes per day” forms.

The invention is thus a modified-release oral pharmaceutical form of losartan comprising a plurality of losartan microunits (mean diameter: 50-1000 μm) making it possible to obtain, after a dose intake, a plasmatic profile of the type shown in FIG. 10.

### FIELD OF THE INVENTION

The field of the present invention is that of oral pharmaceutical forms of losartan, and also treatments and administration methods relating thereto.

### General Aspects

Losartan (or potassium losartan) is an angiotensin II receptor antagonist (type AT1). It is orally active and intervenes in regulating hypertension by acting on the renin-angiotensin system.

Its formula is as follows:
{2-butyl-4-chloro-1-[4-[2-(1H-tetrazol-5-yl)phenyl]benzyl]-1H-imidazol-5-yl}methanol.

Several salts or esters of this product that are pharmaceutically effective and acceptable exist. Thus, throughout the present specification, the term “losartan” denotes losartan per se and/or at least one salt, ester or other pharmaceutically acceptable form thereof.

Losartan may be combined with a diuretic (hydrochlorothiazide) in order to increase its efficacy, or with any other medicament with cardiovascular activity.

Losartan is especially used for treating the following pathologies:

essential arterial hypertension,

treatment of renal insufficiency in the case of type 2 diabetics with proteinuria,

reduction of cardiovascular morbidity and mortality in the case of hypertensive patients with left ventricular hypertrophy (usually in combination with a thiazide diuretic),

congestive heart insufficiency,

polycythemia of kidney transplant patients.

Losartan, administered orally, undergoes extensive first-pass metabolism in the cytochrome P450 enzymatic system. It is partly converted into a carboxylic acid (EXP3174), an active metabolite more powerful than the parent molecule losartan, which is considered, in this respect, as a prodrug.

The definition of “losartan” given above may be advantageously complemented by that mentioned in WO-A-03/035039 (page 3, line 28 to page 4, line 18).

For the purposes of the present specification, the term “losartan” denotes losartan in at least any one of its pharmaceutically acceptable forms, including its metabolites.

Problematics

The assurance of quality and reproducibility of a treatment is a major requirement for any pharmaceutical form, and especially for oral forms of losartan.

However, it may arise that certain oral pharmaceutical forms of losartan do not satisfy this requirement and thus, for the same therapeutic form administered orally at the same dose, certain patients benefit from an adequate and effective therapeutic protection whereas certain others are incorrectly treated and/or, even more seriously, are victims of hazardous side effects.

Such oral pharmaceutical forms of losartan lead to erratic plasmatic profiles and do not guarantee a therapeutic treatment that is homogeneous, effective and tolerable for all patients.

These serious drawbacks are observed for immediate-release oral pharmaceutical forms (IRF) that may be administered one or more times a day.

In particular, it has been observed that the plasmatic concentration profiles obtained after administration of oral IR pharmaceutical forms of losartan result, for 5% to 25% of patients, in an early plasmatic concentration peak of large amplitude, whereas, for the majority of patients, the plasmatic concentration peak (Cmax) occurs later and is of smaller amplitude. This pronounced difference in behavior makes it possible to classify patients into two populations: a population (Pr) with a “rapid” profile and a second population (Ps) with a “slow” profile.

This high variability with premature and massive release of this antihypertensive agent losartan may have serious consequences.

Firstly, patients having an early concentration peak of very large amplitude may suffer from serious hypotension.

Secondly, the early decrease in the plasmatic concentration after the peak is reflected by a very low losartan concentration level, at the end of the period between two administrations. Thus, after having been subjected to a losartan overconcentration corresponding to the peak, the patients of the rapid population Pr are insufficiently treated at the end of the period between two administrations.

Finally, this high variability leads practitioners toward limiting the prescribed doses and certain patients may be incorrectly treated.

It may also prevent the establishment or the administrative authorization of a useful low-dose form of the medicament:

this is the case when, on account of the variability mentioned above for the forms known in the prior art, it proves to be impossible to demonstrate in a manner that satisfies the registrating authorities (double-blind clinical studies against placebo or reference showing a statistically significant difference) that a low-dose form is of therapeutic value for a particular population, for example in the case of children;

another example of such an unsolved problem is that of a low-dose antihypertensive agent for treating mildly expressed forms of hypertension and for which the patients may show occasional episodes of hypotension, for example orthostatic hypotension: a low-dose ARB form might be indicated, provided that it does not risk, via accidentally excessive release, aggravating the risk of hypotension episodes.

It would therefore be advantageous to have available oral pharmaceutical forms of losartan that make it possible to avoid, in particular for the daily administrable forms, erratic behavior of the plasmatic concentration profile with the existence of two populations of patients. In other words, it would be advantageous for the pharmaceutical form to lead to a homogeneous population of plasmatic concentration profiles without massive and/or early and/or rapid release of losartan. This objective is thus distinct from the search for a form with sustained release of losartan.

Moreover, ARBs, especially losartan, are usually administered orally, in tablet form, at a rate of one a day. However, it turns out that such a treatment is not ideal. Specifically, it transpires that the intended control of the arterial pressure is obtained poorly or not at all, or else is obtained in an irreproducible manner. Side effects are also occasionally observed.

The underlying problems are especially the following (1, 2, 3):

1) in most patients, the dosage is 50 mg or 100 mg once a day. However, certain authors [The Angiotensin II type I receptor antagonists: a new class of antihypertensive drugs, Bauer et al., Arch. Inter. Med. 1995, 155, 13, p 1361-1368] describe the taking of a dose of 50 mg twice a day as being more effective than the taking of a single 100-mg dose.

Thus, for about 20% of patients, a daily intake of, for example, 100 mg does not allow an effective treatment, and two dose intakes per day (twice 50 mg) are necessary.

This imperfect performance of a single daily administration is explained by the fact that the arterial pressure of the patients after oral administration of losartan is closely related to the plasmatic concentration of active metabolite E-3174. However, only 12 hours after a single daily dose intake, the plasmatic concentration of active agent E-3174 is already very low. Specifically, the decrease in the plasmatic concentration of E-3174 occurs within 2 to 4 hours of the dose intake; in addition, this decrease is rapid: the half-life of E-3174 being 6-9 hours.

It would thus be recommended to administer this medicament twice a day. However, it is commonly accepted that a dosage involving several intakes per day is not among the most desirable in terms of patient compliance and thus of efficacy of the treatment.

It is thus desirable to have available a form with modified release of losartan that prolongs the bioabsorption time and allows the medicament to be administered only once a day. However, such forms known in the prior art pose the problems outlined hereinbelow.

2) ensuring therapeutic safety is a major challenge for antihypertensives such as losartan. The reason for this is that it is of fundamental importance to have available an oral medicament designed such that, once ingested, the active principle it contains is released into the gastrointestinal tract and bioabsorbed in its absorption window, otherwise the dose of active principle is evacuated with the intestinal transit without being correctly absorbed. It therefore does not produce the expected therapeutic effect. In the case of losartan, the arterial pressure of the patient who has swallowed the tablet is not reduced, which considerably increases the risks of infarction and thus places the patient's life in danger.

The literature describes sustained-release gastro-retentive losartan tablets. The tablet swells in the stomach up to a size which, when the pylorus is closed (i.e. in the fed state), prevents its gastric emptying. The active principle is thus gradually released upstream of its absorption window located in the upper parts of the small intestine. It may thus be absorbed correctly.

However, for a non-negligible proportion of patients, the closing of the pylorus may be erratic. In addition, if the patient does not rigorously adhere to the dosage prescriptions and ingest the tablet before or at the start of a meal before the pylorus has closed, it is possible that the swallowed tablet does not reside in the stomach and is rapidly evacuated without having released the losartan upstream of its bioabsorption window.

These sustained-release gastro-retentive forms are therefore not reliable, since the losartan is not necessarily bioabsorbed, whether the patient is in the fed or fasted state.

It is thus crucial for a modified-release oral form to be able to ensure that, once the oral medicament has been ingested, the losartan is bioabsorbed, whether the patient is in the fed or fasted state.

3) an additional problem arises in the case of modified-release forms of losartan. This active principle has a water solubility that varies greatly as a function of the pH (see the table on page 12 below).

Now, in the fasted state, the gastric pH is 1.4, whereas it is 5 after a meal. Thus, in modified-release forms of losartan for which the release of losartan is either pH dependent (enteric coating), or dependent on the solubility of the active principle, the release kinetics may vary depending on whether or not the patient is fasted.

Consequently, the ideal situation would be to have available an oral pharmaceutical form of losartan:

that leads to a homogeneous population of plasmatic concentration profiles without massive and/or early and/or rapid release of losartan;

that can be administered once a day;

that is more effective than the immediate-release “one dose intake per day” forms;

which is such that once the oral pharmaceutical form has been ingested, the active principle it contains is released into the gastrointestinal tract and bioabsorbed in its gastrointestinal absorption window, whether the patient is in the fed or fasted state, i.e. which allows good reproducibility of the plasmatic concentration, by limiting—or even eliminating—the harmful effects of the interindividual variability of the gastric emptying,

and that overcomes the problem of the variability of the solubility of losartan as a function of the pH.

### PRIOR ART

Monolithic oral pharmaceutical forms and multimicroparticulate oral pharmaceutical forms are known.

### Monolithic Forms

Patent application WO-A-98/24411 describes a therapeutic treatment method using buspirone, which consists in orally administering an immediate-release galenical form (e.g. tablet or gel capsule) comprising both buspirone and a sufficient amount of nefazodone, so as to increase the bioavailability of the buspirone and to reduce its elimination, the formation of metabolite and also the variability of the pharmacokinetic parameters. This combination of nefazodone with buspirone is supposed to overcome the problem that has not been solved by the controlled/sustained-release formulations of buspirone disclosed in U.S. Pat. No. 5,431,922 and which have the major drawback of inducing a high level of variability of the pharmacokinetic parameters (cf. page 3, lines 7 to 16 of WO-A-98/24411).

U.S. Pat. No. 6,248,359 discloses a multitablet system for treating urinary incontinence using oxybutynin. This system comprises a 1^{st} tablet that releases the oxybutynin over a short period of time (e.g. less than 6 hours) and a 2^{nd} tablet that releases oxybutynin over an extended period of time (18 to 24 hours). This system is presented as being able to compensate for the interindividual variability, in response to treatment with oxybutynin. These tablets consist, for example, of tablets each comprising an oxybutynin core and several coats.

The monolithic forms according to WO-A-98/24411 and U.S. Pat. No. 6,248,359 do not concern losartan.

### Multimicroparticulate Forms

Patent application PCT WO-A-96/11675 describes microcapsules for the oral administration of medicinal and/or nutritional active principles (AP), which are less than or equal to 1000 μm in size. These microcapsules consist of particles coated with a coating material, which itself consists of a mixture of a film-forming polymer (ethylcellulose), a hydrophobic plasticizer (castor oil), a surfactant and/or lubricant (magnesium stearate) and a nitrogenous polymer (polyvinylpyrrolidone: PVP). These microcapsules are also characterized by their ability to remain for a long time (at least 5 hours) in the small intestine and to allow, during this residence time, the absorption of the AP over a period longer than the natural transit time in the small intestine.

Patent application PCT WO-A-03/030878 describes a system for the delayed, controlled and definite release of AP, characterized by a twofold mechanism of initiation of release of the AP: “time-dependent” release initiated after a controlled time in the stomach, without change of pH, and “pH-dependent” release initiated by a rise in pH, when the galenical form enters the intestine. These microcapsules with a diameter of between 200 and 600 microns are characterized by a coating film based on a hydrophilic polymer A of Eudragit® L type combined with a hydrophobic compound B, such as a plant wax (Lubritab®) with a melting point of between 40 and 90° C., the ratio B/A being between 0.2 and 1.5.

All these known oral pharmaceutical forms are not presented as offering a guarantee in terms of interindividual reproducibility of the plasmatic concentration profile with elimination of the risk of early and massive release and therapeutic cover over the entire time interval between two dose intakes.

Said oral forms may thus be improved.

To the Inventors' knowledge, the prior art thus lacks any technical proposals liable to provide a start of a solution to this problem of oral pharmaceutical forms leading to erratic plasmatic profiles.

### Objectives

On the basis of these observations, the Inventors set themselves the following objectives.

The essential objective of the invention is to overcome the insufficiencies and drawbacks of the prior art.

Another essential objective of the invention is to propose, in the oral pharmaceutical forms, a novel use of means for controlling the release of losartan (coating or matrix containing losartan) so as to satisfy at least one of the above objectives, and in particular to reduce the interindividual variability of the plasmatic profiles.

Another essential objective of the invention is to propose a novel use of oral pharmaceutical forms comprising means for controlling the release of losartan, of the coating or matrix type containing losartan, so as to satisfy at least one of the above objectives.

Another essential objective of the invention is to propose, in the oral pharmaceutical forms, a novel use of means for controlling the release of losartan (coating or matrix containing losartan), in order to reduce the interindividual standard deviation of the maximum plasmatic after administration.

Another essential objective of the invention is to propose a novel use of oral pharmaceutical forms comprising means for controlling the release of losartan of the coating or matrix type containing losartan, in order to reduce the interindividual variability of the plasmatic profiles and especially to reduce the interindividual standard deviation of the maximum plasmatic concentration after administration.

One essential objective of the invention is to provide an oral pharmaceutical form of losartan, which is used such that it gives access to a more uniform and more reproducible quality of treatment from one patient to another, relative to what is proposed in the prior art.

Another essential objective of the present invention is to propose a means for reducing the interindividual standard deviation of the maximum concentration Cmax of the plasmatic profile.

Another essential objective of the invention is to provide an oral pharmaceutical form of losartan that allows a reduction in the interindividual variability of the plasmatic profiles of the known oral pharmaceutical forms of losartan, in order especially to avoid the appearance of two populations of plasmatic profiles: an at-risk population Pr of “rapid” profiles, and a population Ps of “slow” profiles.

Another essential objective of the present invention is to propose a means for reducing, or even eliminating, the rapid population Pr.

Another essential objective of the invention is to provide an oral pharmaceutical form of losartan that offers assurance in terms of therapeutic safety: elimination of the risk for certain patients of premature and/or massive and/or rapid release of the losartan, and therapeutic cover throughout the time interval between two dose intakes.

Another essential objective of the invention is to provide an oral pharmaceutical form of losartan that guards the patients against any risk of plasmatic overconcentration of the losartan and thus protects them against any medication-related accident.

Another essential objective of the invention is to propose a means for reducing the peak/trough ratio of the plasmatic concentrations of losartan.

Another essential objective of the invention is to provide an oral pharmaceutical form of losartan, which, once ingested, allows the losartan contained to be released into the gastrointestinal tract and bioabsorbed in its absorption window.

Another essential objective of the invention is to provide an oral pharmaceutical form of losartan that can be administered once a day and that is at least as effective as the immediate-release one-a-day forms currently in use.

Another essential objective of the invention is to provide an oral pharmaceutical form of losartan which, when administered once a day, is therapeutically effective, for example for more than 80% of patients.

Another essential objective of the invention is to provide an oral pharmaceutical form of losartan that has an in vitro dissolution profile independent of the dose of losartan.

Another essential objective of the invention is to provide an oral pharmaceutical form of losartan that has the same weight composition irrespective of the intended therapeutic dose of losartan.

Another essential objective of the invention is to provide an oral pharmaceutical form of losartan that can be administered once a day and that is suitable for patients who have difficulty in swallowing, especially children or infants who not only cannot swallow, but also require the administered dose to be adapted as a function of their weight.

Another essential objective of the invention is to provide an oral pharmaceutical form of losartan that can be administered once a day and that offers that possibility of mixing the losartan with one or more active principles in the same oral form, with the possibility of readily and independently adjusting the release times of the various active principles.

Another essential objective of the invention is to provide an oral pharmaceutical form of losartan that can be administered once a day and that limits the risk of deterioration of tissues by local overconcentration of losartan.

Another essential objective of the invention is to provide an oral pharmaceutical form of losartan that can be administered once a day and that, despite the variability of the solubility of losartan in water as a function of the pH, releases the losartan according to the same kinetics, whether or not the patient is fasted.

Another essential objective of the invention is to provide an oral pharmaceutical form of losartan that can exist in various galenical forms, especially including: tablet, sachet, drinkable suspension, gel capsule, and the like.

Another essential objective of the invention is to provide a therapeutic method that consists in using an oral pharmaceutical form that satisfies at least one of the above therapeutic objectives.

All the improvements targeted above are in reference to an immediate-release oral pharmaceutical form of losartan.

### BRIEF DESCRIPTION OF THE INVENTION

In this context, the Applicant has, to its credit:

realized that:
losartan has a solubility that varies greatly as a function of the gastric pH,
and this gastric pH is subject to great variability, the origin of which is varied and dependent on diverse uncontrollable parameters, especially: the fed or fasted state, interindividual variability, action of a medicament influencing these gastrointestinal conditions, etc.

put forward the hypothesis that the irreproducibility of the quality of treatment from one patient to another might be related to the dependence of the solubility of losartan with respect to the gastric pH, which is variable as a function of the time of the dose intake within the same individual and from one individual to another;

and, finally, imagined a technical solution for limiting or even eliminating this dependence, this solution consisting in recommending the use of a coating or matrix containing losartan, which is capable of reducing or even eliminating the rapid plasmatic profile population Pr and of avoiding the premature and/or massive and/or rapid release of the losartan irrespective of the gastric acidity, which is of a nature to reduce the interindividual variability of the plasmatic profiles. In so doing, firstly, the therapeutic safety is improved by preventing the deleterious effects of the oral administration of losartan for a certain population of patients, and, secondly, the therapeutic efficacy is promoted.

Thus, the present invention achieves the above objectives, among others, by proposing:

the use, in an oral pharmaceutical form comprising losartan, of a coating or matrix including said losartan and allowing controlled release of said losartan, such that this form orally administered to a sample of individuals leads, irrespective of the fed or fasted state of the individuals, to a reduction of the interindividual standard deviation of the Cmax, which ensures lower variability of the efficacy and of the therapeutic safety of the pharmaceutical form relative to an immediate-release pharmaceutical form of losartan administered to this same sample of individuals, at the same dose; and/or

the use of losartan contained in a coating or matrix that allows controlled release of said losartan, for manufacturing an oral pharmaceutical form which, after oral administration to a sample of individuals, leads, irrespective of the fed or fasted state of the individuals, to a reduction of the interindividual standard deviation of the Cmax, which ensures lower variability of the efficacy and of the therapeutic safety of the pharmaceutical form relative to an immediate-release pharmaceutical form of losartan administered to this same sample of individuals, at the same dose.

The invention is thus defined by means of a reference clinical test in which the pharmaceutical form is administered orally to a sample of individuals, under experimental conditions which may be those given in the examples below. This clinical test defines the invention by the pharmacokinetic properties specifically obtained under the test conditions. However, the invention is not limited to an implementation under the conditions of this reference clinical test.

The use according to the invention makes it possible to reduce, or even eliminate, the erratic nature of the plasmatic concentration profiles from one individual to another and, in so doing, to avoid:

firstly, the premature release of the AP and thus a plasmatic overconcentration with the side effects thereby entailed, and

secondly, any possible lack of therapeutic cover between two dose intakes.

Thus, the technical function exploited and highlighted in accordance with the invention is not the extension of the release time, but rather the reduction of the variability of the treatment that may be detrimental to the patient. Thus, the invention makes it possible to ensure better efficacy and greater therapeutic safety.

The present invention also proposes a novel modified-release oral pharmaceutical form of losartan, which may be one of those employed in the abovementioned use and defined by claim 31 or 32.

Advantageously, this pharmaceutical form is designed such that the microunits, once ingested, are dispersed and individualized when they reach the stomach, which ensures uniform and gradual gastric emptying of the microunits, in either the fed or fasted state, and thus ultimately a release of losartan in its gastrointestinal bioabsorption window.

For the purposes of the invention, the term “dispersed and individualized” means that the losartan-based microunits are not trapped in a matrix when they arrive into the stomach just after ingestion. The microunits become dispersed in the stomach as soon as they arrive therein (for example in less than two minutes).

### DETAILED DESCRIPTION OF THE INVENTION

The definition of “losartan” given above may advantageously be complemented by that mentioned in WO-A-03/035039 (page 3, line 28 to page 4, line 18).

In the present specification, the term “immediate release” denotes the release by an Immediate-Release Form (IRF) of the majority of the losartan in a relatively short time, for example:
at least 80% is released in vivo in one hour, and preferably in thirty minutes, after oral ingestion;
or at least 80% of the losartan is released in 1 hour, and preferably in thirty minutes, at any pH of between 1.4 and 7.4 in an in vitro dissolution test.

All the dissolution profiles under consideration in the present specification are produced according to the indications of the European Pharmacopea 4^{th} edition entitled: “Test of dissolution of solid oral forms”: type II dissolutest performed under SINK conditions at 37° C. and stirred at 100 rpm.

Examples of such IRFs include standard tablets to be swallowed, dispersible tablets, tablets to be chewed, sachets and gel capsules.

In the present specification, the term “controlled release” denotes a release of losartan by an oral pharmaceutical form, this release taking place in vivo independently of the gastric pH and at a rate lower than that of an “Immediate-Release” Formulation of reference IRF*. Such a controlled-release formulation may comprise, for example, an immediate-release phase and a slow-release phase. Controlled-release formulations are well known in this field; see, for example, Remington: The science and practice of pharmacy, 19^{th} edition, Mack publishing Co. Pennsylvania, USA. The controlled release may especially be a sustained and/or controlled, or even delayed, release.

In the present specification, the term “modified release” denotes a release of losartan by a pharmaceutical formulation, this release taking place at a rate lower than that of an “Immediate-Release” Formulation of reference IRF*, such as a standard tablet or gel capsule to be swallowed. Such a modified-release formulation may comprise, for example, an immediate-release phase and a slow-release phase. Such modified-release formulations are well known in this field: see, for example, Remington: The science and practice of pharmacy, 19th edition, Mack publishing Co. Pennsylvania, USA.

The pharmacokinetic parameters under consideration in the present invention are defined in the following manner. After oral administration of the pharmaceutical form to a sample of N individuals, the individual plasmatic concentration profile is measured on each of the patients, from which are drawn the conventional individual pharmacokinetic parameters: Tmax, Cmax, C24h:

Tmax is the time at which the plasmatic concentration reaches its maximum, Cmax.

C24h is the plasmatic concentration 24 hours after the administration.

From these individual parameters, a person skilled in the art conventionally calculates the mean values of these parameters and their standard deviations. Further details regarding the discussion of these parameters will be found in the book: Pharmacokinetics and Pharmacodynamic Data Analysis 3rd ed., J. Gabrelsson et al., Kristianstads Bocktryckeri AB, Sweden, 2000.

The gastric pH is a magnitude that is intrinsically variable over a pH range of from pH 1.2 to pH 5.5. This variation is observed for the same individual especially according to the fed or fasted state, and from one individual to another. In addition, certain patients may be treated with medicaments that “artificially” modify the gastric pH. This is the case, for example, for proton pump inhibitors (e.g. omeprazole) or antacids.

The Applicant has, to its credit, noted that losartan, whose solubility depends greatly on the gastric pH, leads to erratic plasmatic concentration profiles from one patient to another.

Without the Applicant being able to provide a full explanation for this phenomenon, it may be put forward that this variability of the plasmatic concentration profile results from the variation of the solubility of losartan as a function of the gastric pH. The reason for this is that, in order to be absorbed, the losartan must first be dissolved. This dissolution step thus depends greatly on the gastric pH. Thus, for the same dose of losartan, and according to the gastric pH of the patient, the losartan becomes fully and rapidly dissolved in the case of some patients or, on the contrary, does not dissolve in the stomach in the case of other patients.

It is thus conceived that for losartan, whose solubility depends greatly on the gastric pH (cf. table below), the solubilization, and thus ultimately the plasmatic concentration profile, is subject to great variation from one individual to another and, for the same individual, from one day to another.

| pH | Solubility in g/l at 37° C. |
|---|---|
| 1.4 | 1 |
| 2.0 | 0.6 |
| 3.0 | 0.10 |
| 4.5 | 0.12 |
| 5.0 | 0.3 |
| 6.0 | 1.2 |
| 6.8 | 10 |
| 8.5 | >550 |

Thus, as indicated in example 7 hereinbelow, a conventional IRF of losartan, administered at a dose of 100 mg to a sample of 20 individuals leads to a Cmax that varies from one individual to another by a factor >10 (70 to 800 ng/ml).

This erratic nature of the plasmatic concentration profiles may be reflected, as in the referenced example, by the appearance of two profile populations: the rapid population (Pr) and the slow population (Ps).

For the rapid population, the premature release of losartan has three very detrimental consequences:

(a) the patients of the rapid population are potentially subject to hazardous side effects, such as hypotension, associated with the early plasmatic overconcentration of losartan.
(b) the existence of these risks leads to the prescribed doses being limited, which might deprive certain patients of an adequate treatment.
(c) for the rapid profiles, the plasmatic concentration is very low at the end of the time interval between two administrations. The therapeutic cover of these patients is consequently insufficient.

For the purposes of the invention, the term “rapid population Pr” denotes all the individuals for whom, in the fed state, after administration of an IRF of losartan, the Tmax is less than 1.5 hours. For this rapid population Pr, the maximum plasmatic concentration of losartan is reached early and generally takes a high value.

Thus, one of the essential elements of the invention consists in using or in proposing the use, for therapeutic purposes, of an oral pharmaceutical form comprising losartan contained in a coating or matrix designed to govern the controlled release of the losartan, such that this pharmaceutical form orally administered to a sample of individuals leads, irrespective of the fed or fasted state of the individuals, to a reduction of the interindividual standard deviation of the Cmax, which ensures lower variability of the efficacy and of the therapeutic safety of the pharmaceutical form relative to an immediate-release pharmaceutical form of losartan administered to this same sample of individuals, at the same dose.

The standard deviation reduction factor (f) is defined by the ratio of the standard deviation of the Cmax of the IRF* form to the corresponding standard deviation of the form concerned by the use according to the invention.

Preferably, the reduction factor (f) for the interindividual standard deviation of the Cmax is such that: f≧1.2; preferably f≧1.75 and even more preferentially f is between 2.5 and 20.

The use of said coating or of said matrix for manufacturing such an oral pharmaceutical form is also targeted by the invention.

The oral pharmaceutical form with which said uses is concerned is also a fully fledged subject of the present invention.

The Applicant has thus, to its credit, found that coating losartan in a controlled-release membrane, or including losartan in a controlled-release matrix, makes it possible to eliminate or reduce the erratic nature of the release profiles of losartan from one, individual to another.

This invention appears particularly important for optimizing the use of losartan, which may, by itself, be administered once a day, but which suffers from this erratic behavior of the plasmatic profiles. Thus, the aim of the invention is not principally the extension of the release time, but above all the reduction of the variability of the treatment that may be detrimental to the patient. Thus, the invention makes it possible to ensure better efficacy and therapeutic safety.

In summary, the Applicant's inventive credit is based essentially on the fact that it clearly identified and addressed the problem of the variability of the solubility of losartan according to the gastric pH and the variability of the latter. Starting from these intangible factors, the Applicant has proposed a novel and inventive use of known general means for limiting the influence of these factors. These means are the coating membrane or the inclusion matrix for losartan. They prevent its rapid and early release in the stomach, even in the case of patients whose gastric pH is such that the solubility of losartan is high.

According to another advantageous arrangement of the invention, the pharmaceutical form may be administered daily by means of one or more dosage units of all types (e.g. tablet, gel capsule, volume unit of powder or liquid), with the exclusion of systems including several tablets per dose intake, in which at least one of these tablets is a tablet with rapid release of the active principle (less than 6 hours) and at least one of these tablets is a tablet with sustained release of the same active principle (18 to 24 hours).

The use of a coating on a losartan granule and/or the inclusion of losartan in a matrix makes it possible to attenuate the variations in the solubility of losartan as a function of the pH. Thus, whereas at a pH of between 1 and 3, the solubility varies by a factor of 10, the formulations according to the use in accordance with the invention make it possible to obtain as a function of the time T (in hours) in vitro release profiles for losartan (D, in %) that depend little or not at all on the pH (cf. the attached FIG. 2).

Advantageously, the coating or matrix is designed such that it allows the controlled release of losartan, firstly to avoid any premature and/or massive and/or rapid release of losartan and subsequently any deleterious plasmatic overconcentration of losartan, and secondly to ensure therapeutic cover between two dose intakes.

In accordance with the use according to the invention, the coating or matrix of the pharmaceutical form is designed such that the oral administration of this form, to a sample of individuals, leads to a mean peak/trough modulation of the plasmatic profiles of the metabolite EXP3174 that is less than the mean peak/trough modulation of the metabolite EXP3174 for the same sample of individuals receiving the same dose of an immediate-release form of losartan.

For the purposes of the invention, the peak/trough modulation of the plasmatic concentration profiles is defined by the mean of the ratio Cmax/C24h for the metabolite EXP3174.

In accordance with the use according to the invention, the coating or matrix of the pharmaceutical form is designed such that the oral administration of this form to a sample of individuals leads to a variability of the peak/trough modulation of the plasmatic profiles for the metabolite EXP3174 that is less than the variability of the peak/trough modulation of the metabolite EXP3174 for the same sample of individuals receiving the same dose of an immediate-release form of losartan.

For the purposes of the invention, the variability of the peak/trough modulation of the plasmatic concentration profiles is defined by the standard deviation of the ratio Cmax/C24h for the metabolite EXP3174.

The plasmatic profiles obtained are more uniform. Their interindividual variability is reduced.

In a noteworthy manner, the invention also proposes:

the use, in an oral pharmaceutical form comprising losartan, of a coating or matrix including said losartan and allowing the controlled release of losartan, such that this pharmaceutical form administered orally, to a sample of individuals (for example in the fed state), leads to a decrease in the number or to the disappearance of the individual plasmatic profiles having a Tmax of less than or equal to one hour and preferably less than or equal to 1.5 hours, to the benefit of the individual plasmatic profiles having a Tmax of greater than one hour and preferably greater than 1.5 hours, which ensures lower variability of the efficacy and of the therapeutic safety of the pharmaceutical form, relative to an immediate-release pharmaceutical form of losartan administered to this same sample of individuals (for example in the fed state), at the same dose;

or the use of losartan contained in a coating or matrix allowing the controlled release of losartan, for the manufacture of a pharmaceutical form, which, after oral administration to a sample of individuals (for example in the fed state), leads to a decrease in the number or to the disappearance of the individual plasmatic profiles having a Tmax of less than or equal to one hour and preferably less than or equal to 1.5 hours, to the benefit of the individual plasmatic profiles having a Tmax of greater than one hour and preferably greater than 1.5 hours, which ensures lower variability of the efficacy and of the therapeutic safety of the pharmaceutical form, relative to an immediate-release pharmaceutical form of losartan administered to this same sample of individuals (for example in the fed state), at the same dose.

Comparison of the controlled-release form of losartan used according to the invention and of the immediate-release form (IRF*), and in particular of the pharmacokinetic parameters of their standard deviation, is performed under the same conditions and at the same dose of losartan, on a number of patients, for example greater than or equal to fifteen and preferably greater than or equal to 20.

According to one variant, the invention is also directed toward:

the use, in an oral pharmaceutical form comprising losartan, of a coating or matrix including said losartan, for reducing the variability of the plasmatic profiles during the administration of this pharmaceutical form to a sample of individuals, relative to an immediate-release pharmaceutical form IRF* of losartan administered to this same sample of individuals, at the same dose, which ensures lower variability of the efficacy and of the therapeutic safety of the pharmaceutical form, relative to a pharmaceutical form with immediate release IRF* of losartan administered to this same sample of individuals, at the same dose;

or the use of losartan contained in a coating or matrix, for the manufacture of a pharmaceutical form leading to a reduction in the variability of the plasmatic profiles during the administration of this pharmaceutical form to a sample of individuals, relative to a pharmaceutical form with immediate release IRF* of losartan administered to this same sample of individuals, at the same dose, which ensures lower variability of the efficacy and of the therapeutic safety of the pharmaceutical form, relative to an immediate-release pharmaceutical form of losartan administered to this same sample of individuals, at the same dose.

The pharmaceutical form targeted in the use according to the invention may contain losartan in the form of microunits, which may especially be:

microparticles individually consisting of a core that comprises losartan and that is coated with at least one coating allowing the controlled release of the losartan (also referred to hereinbelow as coated microparticles);

and/or microgranules individually consisting of a matrix that includes losartan and allows the controlled release of the losartan (also referred to hereinbelow as matrix microgranules);

and/or immediate-release losartan microgranules.

The oral pharmaceutical form targeted in the use according to the invention may be any of the forms known to those skilled in the art, i.e. especially gel capsules, sachets, suspensions containing losartan microunits or tablets.

These tablets may be

(i) either tablets containing losartan microunits,

(ii) or tablets free of microparticles individually consisting of a core comprising losartan and being coated with at least one coating allowing the controlled release of the losartan, and/or free of microgranules individually consisting of a matrix including losartan and allowing the controlled release of the losartan.

These tablets (ii) may be matrix tablets or individually coated tablets.

The losartan microunits (controlled-release coated microparticles and/or matrix microgranules or immediate-release microgranules) preferably have a mean diameter (Dm in μm) of less than 1000, preferably between 50 and 800 and even more preferentially between 50 and 500.

Advantageously, the oral pharmaceutical form intended in the use according to the invention makes it possible to obtain, after a dose intake, a plasmatic profile defined as follows:

| | Cmax/C24 h ≦ Cmax*/C24 h* |
|---|---|
| preferably | 1.5 × Cmax/C24 h ≦ Cmax*/C24 h* |
| and even more preferentially | 2.0 × Cmax/C24 h ≦ Cmax*/C24 h* |

with:

C24h representing the mean plasmatic concentration of active metabolite EXP3174 of losartan, 24 hours after the dose intake,

C24h* representing the mean plasmatic concentration of EXP3174 obtained under the same conditions as C24h, with a reference immediate-release oral pharmaceutical form, containing the same dose of losartan,

Cmax representing the mean maximum plasmatic concentration of EXP3174 after the dose intake,

Cmax* representing the mean maximum plasmatic concentration of EXP3174 obtained under the same conditions as Cmax, with a reference immediate-release oral pharmaceutical form, containing the same dose of losartan.

In accordance with a first embodiment, the oral pharmaceutical form comprises coated or matrix microparticles and has an in vitro dissolution profile [D % (t)] such that: the time t(70%) after the administration and after which 70% of the losartan is released is between 1 and 24 hours, preferably between 2 and 12 hours and even more preferentially between 2 and 8 hours.

According to one advantageous characteristic of this first embodiment, for any value of the time t between 2 hours and t(70%), preferably between 1 hour and t(70%), the percentage of dissolved (released) losartan [D % (t)]≧35×t/t(70%).

Preferably, the oral pharmaceutical form according to the first embodiment is characterized in that the rate of release of the losartan in vitro, in a dissolution test, is independent of the pH.

The pH values more specifically concerned are the physiological pHs in the stomach, for example those ranging from 1 to 7.

The composition of the individual coating or of the individual matrix of the microparticles according to the first embodiment advantageously corresponds to one of the following two families A and B:

Family A

A-1—at least one film-forming polymer (P1) that is insoluble in the fluids of the tract, present (weight % of solids) in a proportion of from 50 to 90 and preferably 50 to 80 relative to the total mass of the coating composition and especially comprising at least one water-insoluble cellulose derivative;

A-2—at least one nitrogenous polymer (P2) present (weight % of solids) in a proportion of from 2 to 25 and preferably 5 to 15 by weight of solids relative to the total mass of the coating composition and consisting of at least one polyacrylamide and/or one poly-N-vinylamide and/or one poly-N-vinyllactam;

A-3—at least one plasticizer present (weight % of solids) in a proportion of from 2 to 20 and preferably from 4 to 15 by weight of solids relative to the total mass of the coating composition and consisting of at least one of the following compounds: glycerol esters, phthalates, citrates, sebacates, cetyl alcohol esters, castor oil;

A-4—at least one surfactant and/or lubricant, present (weight % of solids) in a proportion of from 2 to 20 and preferably from 4 to 15 by weight of solids relative to the total mass of the coating composition and chosen from anionic surfactants and/or from nonionic surfactants and/or from lubricants; said agent possibly comprising only one or a mixture of the abovementioned products.

Examples of compounds A-1, A-2, A-3 and A-4 are cited below:

A-1: ethylcellulose and/or cellulose acetate;

A-2: polyacrylamide and/or polyvinylpyrrolidone;

A-3: castor oil;

A-4: alkali metal or alkaline-earth metal salt of fatty acids, stearic acid and/or oleic acid being preferred, polyoxyethylenated ester of sorbitan, polyoxyethylenated castor oil derivatives, stearates, preferably calcium, magnesium, aluminum or zinc stearate.

Family B:

B1—at least one film-forming polymer that is insoluble in the fluids of the gastrointestinal tract,

B2—at least one water-soluble polymer,

B3—at least one plasticizer,

B4—and optionally at least one surfactant/lubricant preferably consisting of at least one anionic surfactant and/or at least one nonionic surfactant.

Examples of compounds B1, B2, B3 and B4 are cited below:

B1:

water-insoluble cellulose derivatives, ethylcellulose and/or cellulose acetate being particularly preferred,

acrylic derivatives,

polyvinyl acetates,

and mixtures thereof.

B2:

water-soluble cellulose derivatives,

polyacrylamides,

poly-N-vinylamides,

poly-N-vinyllactams,

polyvinyl alcohols (PVA),

polyoxyethylenes (POE), polyvinylpyrrolidones (PVP) (the latter being preferred),

and mixtures thereof;

B3:

glycerol and esters thereof, preferably in the following subgroup: acetylated glycerides, glyceryl monostearate, glyceryl triacetate, glyceryl tributyrate,

phthalates, preferably in the following subgroup: dibutyl phthalate, diethyl phthalate, dimethyl phthalate, dioctyl phthalate,

citrates, preferably in the following subgroup: acetyl tributyl citrate, acetyl triethyl citrate, tributyl citrate, triethyl citrate,

sebacates, preferably in the following subgroup: diethyl sebacate, dibutyl sebacate,

adipates,

azelates,

benzoates,

plant oils,

fumarates, preferably diethyl fumarate,

malates, preferably diethyl malate,

oxalates, preferably diethyl oxalate,

succinates, preferably dibutyl succinate,

butyrates,

cetyl alcohol esters,

salicylic acid,

malonates, preferably diethyl malonate, castor oil (this being particularly preferred), and mixtures thereof;

B4:

alkali metal or alkaline-earth metal salts of fatty acids, stearic acid and/or oleic acid being preferred,

polyoxyethylenated oils, preferably polyoxyethylenated hydrogenated castor oil,

polyoxyethylene-polyoxypropylene copolymers,

polyoxyethylenated sorbitan esters,

polyoxyethylenated castor oil derivatives,

stearates, preferably calcium, magnesium, aluminum or zinc stearate,

stearylfumarates, preferably sodium stearylfumarate,

glyceryl behenate,

and mixtures thereof.

Preferably, the coating consists of only one coat, the mass of which represents from 1% to 50% by weight and preferably from 5% to 40% by weight of the total mass of the microparticles.

Other details and examples of compositions and processes for obtaining microparticles according to the first embodiment according to the invention are given in WO-A-03/084 518, the content of which is incorporated into the present specification by reference.

For further qualitative and quantitative details regarding the coating compositions of families A1 & A2, reference will be made, respectively, to European patent EP-B-0 709 087 and to patent application WO-A-2004/010 984, the content of which is incorporated into the present specification by reference.

In accordance with a second embodiment, the oral pharmaceutical form firstly comprises coated microparticles and/or matrix microparticles, and secondly is such that:

the release of the losartan is governed by two different initiation mechanisms, one being based on a pH variation and the other allowing the release of the losartan after a predetermined residence time in the stomach;

at a constant pH of 1.4, the dissolution profile comprises a lag phase lasting less than or equal to 7 hours, preferably less than or equal to 5 hours and even more preferentially between 1 and 5 hours,

and the passage from pH 1.4 to pH 7.0 leads to a release phase starting without any lag time.

Advantageously, the oral pharmaceutical form according to this second mode has a dissolution profile, measured in an in vitro dissolution test, as indicated below:

less than 20% of the losartan is released after 2 hours at pH 1.4;

at least 50% of the losartan is released after 16 hours at pH 1.4.

According to another preferred characteristic, the oral pharmaceutical form according to this second mode comprises controlled-release losartan microparticles whose initiating pH value is between 6.0 inclusive and 6.5 inclusive.

Advantageously, the controlled-release losartan microparticles according to the second embodiment have the following specific features:

the coating or matrix allowing the controlled release of the losartan comprises a composite material
comprising:
   at least one hydrophilic polymer I bearing groups that are ionized at neutral pH,
   at least one hydrophobic compound II;
representing a mass fraction (weight % relative to the total mass of the microparticles)≦40; and

their mean diameter is less than 1000 μm, preferably between 50 and 800 μm and even more preferentially between 50 and 500 μm.

According to another advantageous characteristic, the composite material I-II of the coating or of the matrix allowing the controlled release of losartan is such that:

the weight ratio II/I is between 0.2 and 1.5 and preferably between 0.5 and 1.0,

and the hydrophobic compound II is selected from products which are crystalline in the solid form and which have a melting point T_{fII}≧40° C., preferably T_{fII}≧50° C. and even more preferentially 40° C.≦T_{fII}≦90° C.

According to one preferred embodiment, the hydrophilic polymer I is chosen from:

I.a copolymers of (meth)acrylic acid and of an alkyl ester of (meth)acrylic acid, and mixtures thereof;

I.b cellulose derivatives, preferably cellulose acetates, cellulose phthalates, cellulose succinates, and mixtures thereof, and even more preferentially hydroxypropylmethylcellulose phthalates, hydroxypropylmethylcellulose acetates and hydroxypropylmethylcellulose succinates, and mixtures thereof;

and mixtures thereof.

The polymers I that are even more preferred are copolymers of (meth)acrylic acids and of alkyl esters (e.g. C1-C6 alkyl) of (meth)acrylic acid. These copolymers are, for example, of the type sold by the company Röhm Pharma Polymers under the registered trade marks Eudragit®, of the series L and S (for instance Eudragit® L100, S100, L30 D-55 and L100-55). These copolymers are anionic enteric copolymers that are soluble in aqueous medium at pH values above those encountered in the stomach.

Still according to the preferred embodiment, compound II is chosen from the following group of products:

II.a plant waxes taken alone or as mutual mixtures;
II.b hydrogenated plant oils taken alone or as a mutual mixture;
II.c glyceryl mono- and/or di- and/or triesters of at least one fatty acid;
II.d mixtures of glyceryl monoesters, diesters and triesters of at least one fatty acid;
II.e and mixtures thereof.

Even more preferably, compound II is chosen from the following group of products: hydrogenated cotton seed oil, hydrogenated soybean oil, hydrogenated palm oil, glyceryl behenate, hydrogenated castor oil, tristearine, tripalmitine, trimyristine, yellow wax, hard fatty substance or fat useful as suppository bases, anhydrous dairy fats, lanolin, glyceryl palmitostearate, glyceryl stearate, lauryl macrogol glycerides, cetyl alcohol, polyglyceryl diisostearate, diethylene glycol monostearate, ethylene glycol monostearate, Omega 3 and any mixture thereof,

preferably from the following subgroup of products: hydrogenated cotton seed oil, hydrogenated soybean oil, hydrogenated palm oil, glyceryl behenate, hydrogenated castor oil, tristearine, tripalmitine, trimyristine and any mixture thereof.

In practice, and without this being limiting, it is preferable for compound II to be chosen:

from the group of products sold under the following brand names: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol Diisostéarique®, Geleol®, Hydrine®, Monthyle® and mixtures thereof;

and also from the group of additives whose codes are as follows: E 901, E 907, E 903 and mixtures thereof;

and preferably from the group of products sold under the following brand names: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab® and mixtures thereof.

According to another advantageous characteristic of the invention, the coating or the matrix allowing the controlled release of losartan is free of talc.

Advantageously, the coating or the matrix of the microparticles may comprise, besides the essential constituents I and II, other standard ingredients known to those skilled in the art, especially such as:

dyes,

plasticizers, for instance dibutyl sebacate,

hydrophilic compounds, for instance cellulose and derivatives thereof or polyvinylpyrrolidone and derivatives thereof,

and mixtures thereof.

Without this being limiting and according to an even more preferred embodiment, the coating of the coated controlled-release losartan microparticles comprises only one composite coating film I-II.

Other details and examples of compositions and processes for obtaining the microparticles according to the second embodiment of the invention are given in WO-A-03/030 878, the content of which is incorporated into the present specification by reference.

In quantitative terms, the coating monolayer may represent, for example, not more than 40% and preferably not more than 30% by weight of the microparticles. Such a limited content of coating makes it possible to produce galenical units each containing a high dose of losartan, without exceeding a size that is prohibitive with regard to swallowing. The compliance with the treatment and thus the success of the treatment are undoubtedly improved thereby.

According to a third embodiment, the oral pharmaceutical form comprises at least two populations of microparticles. Each population of controlled-release losartan microparticles may be in accordance with the first or the second embodiment presented above.

According to one variant -2i- of the second embodiment combined with the third embodiment, the oral pharmaceutical form implemented in the use according to the invention comprises at least two populations of microparticles with different dissolution profiles, for at least one pH value of between 1.4 and 7.4.

According to one variant -2ii- of the second embodiment combined with the third embodiment, the oral pharmaceutical form implemented in the use according to the invention comprises at least two populations of controlled-release losartan microparticles that differ in their respective initiating pH values.

According to yet another variant -2iii- of the second embodiment combined with the third embodiment, the oral pharmaceutical form according to the invention comprises at least two populations of controlled-release losartan microparticles that differ in their respective initiating times.

According to a fourth embodiment, the oral pharmaceutical form implemented in the use according to the invention comprises at least one population of controlled-release losartan microparticles and at least one population of immediate-release losartan microgranules.

According to one variant -2iv- of the second embodiment combined with the fourth embodiment, the oral pharmaceutical form implemented in the use according to the invention comprises:

at least one population of immediate-release losartan microgranules;

at least one population P₁ of controlled-release losartan microparticles, and

at least one population P₂ of controlled-release losartan microparticles;

and, moreover, the respective initiating pH values of P₁ and P₂ differ by at least 0.5 pH unit, preferably by at least 0.8 pH unit and even more preferentially by at least 0.9 pH unit.

Advantageously, the respective initiating pH values of the various populations of controlled-release losartan microparticles are between 5 and 7.

According to one variant -2v- of the second embodiment combined with the fourth embodiment, the oral pharmaceutical form implemented in the use according to the invention comprises:

at least one population of immediate-release losartan microgranules;

at least one population P₁′ of controlled-release losartan microparticles, the initiating pH value of which is equal to 5.5; and

at least one population P₂′ of controlled-release losartan microparticles, the initiating pH value of which is between 6.0 inclusive and 6.5 inclusive.

The populations P₁, P₂, P₁′ and P₂′ of the variants -2iv- and -2v- of the 2^{nd} embodiment comprise controlled-release losartan microparticles, obtained in accordance with the 2^{nd} embodiment.

To illustrate the variants of the invention according to which the immediate-release losartan microunits are present in the oral pharmaceutical form implemented in the use according to the invention, it may be pointed out that these variants may correspond to the case where this pharmaceutical form comprises, for example, at least one population of immediate-release losartan microgranules.

When the oral pharmaceutical form implemented in the use according to the first embodiment comprises controlled-release losartan microparticles, the dissolution profiles of said microparticles between pH 1 and pH 5 are similar, according to the similarity factor f2 calculated as indicated in the FDA directive SUPAC-MR—Modified Release Solid Oral Dosage Forms, i.e. as soon as f2≧50%.

The oral pharmaceutical forms employed in the use according to the invention may comprise at least one active principle other than losartan. The abbreviation AP will denote hereinbelow, without discrimination, one or more active principles other than losartan.

The in vivo or in vitro release of the AP may be immediate or controlled. The AP may be contained in microunits of microgranule type with immediate release of the AP or in microparticles with controlled release of the AP.

This AP may be chosen, inter alia, from the group comprising diuretics, beta-blockers, inhibitors, sodium-channel blockers, alpha-blockers, alpha-beta-blockers, vasodilators, alpha-antagonists and adrenergic neuronal blockers.

For further details regarding these additional APs, reference may be made, for example, to the passage on page 4, line 19-page 4, line 31 of WO-A-03/035 039.

The above-targeted coated microparticles may have several structures.

Thus, according to a first form of structure of the coated microparticles, at least some of the controlled-release losartan microparticles of the oral pharmaceutical form each comprise:

a core containing losartan and

at least one coating covering the core and allowing the controlled release of said losartan.

According to a second form of structure of the coated microparticles, at least some of said controlled-release losartan microparticles of the oral pharmaceutical form each comprise:

a core comprising:
a neutral core, and
at least one active layer comprising the losartan and coating the neutral core,

and at least one coating covering the core and allowing the controlled release of the losartan.

Advantageously, the proportion of losartan in the microunits (expressed as a weight percentage of solids relative to the total mass of the microunits) is between 5 and 80, preferably between 10 and 75 and even more preferentially between 15 and 70. According to one possibility, the immediate-release losartan microgranules are uncoated cores of controlled-release losartan microparticles.

As indicated previously, the invention is also directed toward a specific group of oral pharmaceutical forms among those generally described above. The pharmaceutical forms of the specific group are those comprising microunits formed from coated losartan microparticles. Each pharmaceutical form of this specific group is a modified-release oral pharmaceutical form of losartan as characterized in claim 31 or 32.

This pharmaceutical form as claimed in claim 31 is designed such that the microunits, once ingested, are dispersed and individualized when they reach the stomach, which ensures uniform and gradual gastric emptying of the microunits, in either the fed or fasted state, and thus ultimately release of losartan in its gastrointestinal bioabsorption window.

In the context of the description of the pharmaceutical form as claimed in claim 31 or 32:

the microunits denote:
microparticles coated with at least one coating allowing the modified release of losartan, and
immediate-release losartan microgranules;
with the exclusion of microgranules individually consisting of a matrix that includes losartan and that allows the controlled release of losartan (also referred to hereinbelow as matrix microgranules);

the expression “dispersed and individualized” means that the losartan-based microunits are not trapped in a matrix when they enter the stomach just after ingestion. The microunits become dispersed in the stomach as soon as they arrive therein (for example in less than two minutes).

The advantages of the pharmaceutical form as claimed in claim 31 or 32 are especially the following:

this oral pharmaceutical form of losartan, which may be administered once a day, ensures that once the oral pharmaceutical form has been ingested, the losartan it contains is released into the gastrointestinal tract and bioabsorbed in its absorption window;

this oral pharmaceutical form of losartan, which may be administered once a day, ensures that once the oral pharmaceutical form has been ingested, the losartan it contains will not pass before its bioabsorption window without being released;

this oral pharmaceutical form of losartan, which may be administered once a day, ensures that once the oral pharmaceutical form has been ingested, the losartan it contains will be released independently of the open or closed state of the pylorus;

this oral pharmaceutical form of losartan, which may be administered once a day, is at least as effective as the immediate-release one-a-day forms currently in use;

this oral pharmaceutical form of losartan, which may be administered once a day, is not or only very little subject to the phenomenon of interindividual variability of the gastric emptying and ultimately of the in vivo absorption of losartan and of its active metabolite E3174;

this oral pharmaceutical form of losartan is therapeutically effective, for example for more than 80% of patients, when it is administered once a day;

this oral pharmaceutical form of losartan, which may be administered once a day and which comprises modified-release losartan microunits, draws part of its advantages from the small size (50-1000 μm) of these microunits and their large number (e.g. several thousand per dose), which allows gradual and well-controlled gastric emptying, independently of the taking of food by the patients;

this oral pharmaceutical form of losartan, which may be administered once a day, makes it possible to increase the Tmax of losartan and also the period for which the plasmatic losartan concentration is higher than the plasmatic baseline losartan concentration below which losartan is therapeutically ineffective;

this oral pharmaceutical form of losartan has an in vitro dissolution profile that is independent of the dose of losartan;

this oral pharmaceutical form of losartan may have the same weight composition irrespective of the dose of losartan it contains;

this oral pharmaceutical form of losartan, which may be administered once a day, is suitable for patients who have difficulty in swallowing, especially children or infants who not only cannot swallow, but also need the administered dose to be adapted as a function of their weight;

this oral pharmaceutical form of losartan, which may be administered once a day, offers the possibility of mixing the losartan with one or more other active principles in the same oral form, the respective release times of these various active principles possibly being readily adjusted independently of each other;

this oral pharmaceutical form of losartan, which may be administered once a day, despite the variability of the solubility of losartan in water as a function of the pH, can release the losartan according to the same kinetics, whether or not the patient is fasted;

this oral pharmaceutical form of losartan may exist in various galenical forms, especially including: tablet, sachet, drinkable suspension, gel capsule, etc.;

the oral galenical form according to the invention is composed of a large number (for example from about one thousand to several thousand) of microunits (losartan microparticles or microgranules), this multiplicity statistically ensuring good reproducibility of the losartan transit kinetics throughout the gastrointestinal tract and consequently good control of the bioavailability and better efficacy;

the use of a mixture of microparticles with different modified-release profiles makes it possible to produce release profiles having several waves of release or ensuring, by means of suitable adjustment of the various fractions, a constant level of plasmatic concentration of losartan;

the sensitivity to the variability of the gastric emptying is reduced, since the emptying, which takes place in this case on a large number of particles, is statistically more reproducible;

the placing in contact of tissues with a high dose of losartan (“dose dumping”) is avoided. Specifically, each microunit contains only a very small dose of losartan. This therefore overcomes the risk of deterioration of tissues by local overconcentration of corrosive losartan;

this pharmaceutical form does not induce any degradation of the losartan and preserves the polymorphism of the starting losartan;

their size of between 50 and 1000 μm and also the characteristics of their coating, where appropriate, allows the microunits to increase their transit time in the upper parts of the gastrointestinal tract, which ensures an increase in the time of passage of losartan before its absorption window and thus maximizes the bioavailability of losartan.

Preferably, at least some of the microunits of this pharmaceutical form as claimed in claim 31 or 32 are microparticles individually composed of a core that comprises losartan and that is coated with at least one coating allowing the modified release of the losartan.

As regards the microunits of microparticle type of this pharmaceutical form as claimed in claim 31 or 32, it should be noted that their coating that controls the modified release of losartan comprises essentially pharmaceutically acceptable excipients.

It may also be very advantageous for at least some of the microunits of the pharmaceutical form as claimed in claim 31 or 32 to be composed of immediate-release losartan microgranules.

Preferably, the pharmaceutical form as claimed in claim 31 or 32 is characterized in that the variability CV (in %) of the area under the curve (AUC) of the plasmatic concentration of active metabolite E3174, as a function of the time (T) after dose intake, is less than or equal to 200%, preferably 150% and even more preferentially 120% of the corresponding variability CV* (in %) of the area under the curve (AUC*) of the plasmatic concentration of active metabolite E3174, as a function of the time (T) after dose intake under the same conditions, of a reference immediate-release oral pharmaceutical form*, containing the same dose of losartan, i.e.: CV≦1.5×CV* and preferably CV≦1.2×CV*.

The pharmacokinetic parameters CV and AUC are well known to those skilled in the art.

The comparison of the modified-release form of losartan according to the invention and of the IRF*, and in particular of the parameters CV and CV*, AUC and AUC*, is performed in a statistically significant manner, under the same conditions and at the same dose of losartan.

All the in vitro dissolution profiles concerned in the present specification are performed according to the indications of the European pharmacopea 4th edition entitled: “Test of the dissolution of solid oral forms”: type II dissolutest performed under SINK conditions maintained at 37° C. and stirred at 100 rpm.

In accordance with a first embodiment, the pharmaceutical form as claimed in claim 31 or 32 has an in vitro dissolution profile such that: 70% of the losartan is released between 1 and 24 hours, preferably between 2 and 12 hours and even more preferentially between 2 and 8 hours after administration.

Advantageously, the pharmaceutical form as claimed in claim 31 or 32, in its first embodiment, is characterized in that the rate of release of losartan in vitro in a dissolution test is independent of the pH.

The pH values more specifically concerned are the gastric physiological pH values, for example those ranging from 1 to 7.

This noteworthy property of the pharmaceutical forms as claimed in claim 31 in their first embodiment is all the more advantageous for losartan since the latter is known for its solubility in water that is highly dependent on the pH, as has been recalled hereinabove.

The composition for coating (coating film) the microparticles as claimed in claim 31 or 32, in their first embodiment, advantageously corresponds to one of the two families A and B as defined above.

The coating (coating film) for the microparticles as claimed in claim 31 or 32, in their first embodiment, is in accordance with that described above for the general family of microunits according to the invention.

This is likewise the case as regards the details and examples of compositions and processes for obtaining these microparticles as claimed in claim 31 or 32, in their first embodiment, and also as regards the qualitative and quantitative data for the coating composition of family A.

In accordance with a second embodiment, the pharmaceutical form as claimed in claim 31 is such that:

the release of losartan is governed by two separate initiating mechanisms, one being based on a pH variation and the other allowing the release of the AP, after a predetermined residence time in the stomach;

at a constant pH of 1.4, the dissolution profile comprises a lag phase with a duration of less than or equal to 7 hours, preferably less than or equal to 5 hours and even more preferentially between 1 and 5 hours,

and passing from pH 1.4 to pH 7.0 leads to a release phase starting without a lag time.

For a more detailed description of this second embodiment, reference will be made to the above description of the second embodiment of the microunits of the general family according to the invention.

Similarly, everything that has been indicated hereinabove in reference to the microunits of the general family according to the invention from “the third embodiment” up to the possibility according to which “the immediate-release losartan microgranules are uncoated cores of modified-release losartan microparticles” is directly transposable and applicable to the pharmaceutical form as claimed in claim 31 or 32.

Losartan exists in several crystalline forms, one of which is particularly crucial for the pharmaceutical activity, namely the crystalline form I. It is important that the latter be preserved. Thus, the process used for preparing the pharmaceutical form according to the invention makes it possible to conserve losartan in its initial crystalline form. In the pharmaceutical form according to the invention, for example, at least 50% of the losartan is in its crystalline form I.

As regards the preparation of the coated microparticles according to the invention, this relates to microencapsulation techniques that are accessible to those skilled in the art, the principles of which are summarized in the article by C. Duverney and J. P. Benoit in “L'actualité chimique”, December 1986. More specifically, the technique under consideration is microencapsulation by film coating, leading to individualized “reservoir” systems as opposed to matrix systems.

For further details, reference will be made to patent EP-B-0 953 359.

The losartan particles of desired granulometry necessary for producing the microparticles according to the invention may be pure losartan crystals and/or crystals that have undergone a pretreatment via one of the conventional techniques of the art, for instance granulation, in the presence of at least one standard binder and/or an agent for modifying the intrinsic solubility characteristics of losartan. The losartan may be, for example, deposited on the core via techniques known to those skilled in the art, for instance the “spray coating” technique in a fluidized air bed or formed by wet granulation, compacting, extrusion-spheronization, etc.

Advantageously, the oral pharmaceutical form implemented in the use according to the invention is in a single daily oral dose form comprising from 1000 to 500 000 microunits containing losartan.

More specifically, the oral pharmaceutical form implemented in the use according to the invention may be in a single daily oral dose form comprising from 1000 to 500 000 controlled-release losartan microparticles.

The oral pharmaceutical form according to the invention may be provided in the form of a sachet of powder of controlled-release losartan microparticles, a liquid suspension of controlled-release losartan microparticles, a tablet possibly containing controlled-release losartan microparticles, or a gel capsule containing controlled-release losartan microparticles.

A subject of the present invention is also the oral pharmaceutical form as described above in the context of the use according to the invention and taken as such independently of the envisioned use.

According to another of its objects, the invention is directed toward the use of controlled-release losartan microparticles as defined above and optionally immediate-release losartan microgranules as defined above, for the preparation of microparticulate pharmaceutical or dietetic oral galenical forms, preferably in the form of tablets that are advantageously orodispersible, or powders or gel capsules.

According to yet another of its objects, the invention is directed toward the use of controlled-release losartan microparticles and/or microgranules as defined above and optionally immediate-release losartan microgranules as defined above, for the preparation of a therapeutically safe microparticulate oral pharmaceutical form, designed such that once said pharmaceutical form has been ingested, the microparticles it comprises are dispersed and individualized when they reach the stomach, which allows these microparticles to undergo uniform and gradual gastric emptying, whether the patient is fed or fasted during the dose intake, thus ensuring release of the losartan in its gastrointestinal bioabsorption window, which may participate toward reducing the variability of the plasmatic profiles of losartan.

According to yet another of its subjects, the invention is directed toward coated microparticles and/or matrix microgranules per se as defined above.

According to other subjects thereof, the invention is directed toward:

a therapeutic method for treating hypertension, characterized in that it consists in administering, preferably as a single daily oral dose, the pharmaceutical form implemented in the use according to the invention as defined above;

a reproducible method for human or animal oral therapeutic treatment, characterized in that it consists essentially in administering orally a pharmaceutical form comprising losartan contained in a coating or matrix including said losartan and allowing the controlled release of said losartan, such that this form administered orally to a sample of individuals leads, irrespective of the fed or fasted state of the individuals, to a reduction in the interindividual standard deviation of the Cmax, which ensures lower variability of the efficacy and of the therapeutic safety of the pharmaceutical form, relative to an immediate-release losartan pharmaceutical form administered to this same sample of individuals, at the same dose;

a reproducible method for human or animal oral therapeutic treatment, characterized in that it consists essentially in administering orally a pharmaceutical form comprising losartan contained in a coating or matrix that imparts to this pharmaceutical form properties such that the oral administration of this pharmaceutical form, in the fed state, to a sample of individuals, leads to a reduction in the number or to the disappearance of the individual plasmatic profiles having a Tmax of less than or equal to one hour and preferably less than or equal to 1.5 hours, to the benefit of the individual plasmatic profiles having a Tmax of greater than one hour and preferably greater than 1.5 hours, which ensures lower variability of the efficacy and of the therapeutic safety of the pharmaceutical form, relative to an immediate-release losartan pharmaceutical form administered to this same sample of individuals;

a reproducible method for human or animal oral therapeutic treatment, characterized in that it consists essentially in administering orally a pharmaceutical form comprising losartan contained in a coating or matrix, to reduce the variability of the plasmatic profiles during the oral administration of this pharmaceutical form to a sample of individuals, relative to an immediate-release pharmaceutical form of losartan administered to this same sample of individuals, which ensures lower variability of the efficacy and of the therapeutic safety of the pharmaceutical form;

a reproducible method for human or animal oral therapeutic treatment, characterized in that it consists essentially in administering orally a pharmaceutical form comprising losartan whose solubility is dependent on the gastric pH, the losartan being contained in a coating or matrix that imparts to this pharmaceutical form properties such that the oral administration of this pharmaceutical form to a sample of individuals leads to a reduction in the coefficient of interindividual variation of the Tmax, relative to an immediate-release pharmaceutical form of losartan administered to this same sample of individuals, at the same dose, which ensures lower variability of the efficacy and of the therapeutic safety of the pharmaceutical form.

### DESCRIPTION OF THE FIGURES

FIG. 1 represents the in vitro dissolution profile at pH 6.8 of the controlled-release losartan microparticles according to example 2;

FIG. 2 represents the in vitro dissolution profile at pH 1.4, 4.5 and 6.8 of the controlled-release losartan microparticles according to example 2;

FIG. 3 represents the in vitro dissolution profile at pH 6.8 of the controlled-release losartan microparticles according to example 2 for losartan doses ranging between 10 and 200 mg;

FIG. 4 represents the in vitro dissolution profile at pH 1.4 and 6.8 of the controlled-release losartan microparticles according to example 3;

FIG. 5 represents the in vitro dissolution profile at pH 1.4 and 6.8 of the controlled-release losartan microparticles according to example 4;

FIG. 6 represents the in vitro dissolution profile at pH 1.4 of the controlled-release losartan microparticles according to example 4 for losartan doses ranging between 10 and 200 mg;

FIG. 7 represents the in vitro dissolution profile at pH 6.8 of the controlled-release losartan microparticles according to example 5;

FIG. 8 represents the individual pharmacokinetic profiles of losartan after administration of the formulation C1, which is not included in the context of the invention. The existence of a rapid population Pr and of a slow population Ps will be noted in this FIG. 8;

FIG. 9 represents the individual pharmacokinetic profiles of EXP3174 after administration of the formulation C1, which is not included in the context of the invention. The existence of a rapid population Pr and of a slow population Ps will be noted in this FIG. 8;

FIG. 10 represents the individual pharmacokinetic profiles of losartan after administration of the formulation M1 according to example 2, which is included in the context of the invention. It will be noted that this formulation M1 leads to a single slow population Ps of plasmatic concentration profiles;

FIG. 11 represents the individual pharmacokinetic profiles of EXP3174 after administration of the formulation M1 according to example 2;

FIG. 12 represents the individual pharmacokinetic profiles of losartan after administration of the formulation M2 according to example 4;

FIG. 13 represents the individual pharmacokinetic profiles of EXP3174 after administration of the formulation M2 according to example 4.

Throughout the figures, the dissolution profile corresponds to the weight percentage of losartan dissolved (D) as a function of the time (t) in hours.

### EXAMPLES

In the examples that follow, the excipients are denoted as their trade name. The correspondence with the chemical name will be found in the table below:

| Trade name | Chemical name/Monograph |
|---|---|
| Cremophor RH 40 | Macrogolglycerol hydroxystearate |
| Klucel EF | Hydroxypropylcellulose |
| Plasdone K29/32 | Povidone |
| Eudragit L100-55 | Poly(methacrylic acid, ethyl acrylate) 1:1 |
| Eudragit S100 | Poly(methacrylic acid, methyl methacrylate) 1:2 |

### Example 1

### Preparation of Potassium Losartan Granules

810 g of potassium losartan and 90 g of Klucel EF® (Aqualon) are dispersed in 3000 g of isopropanol. The suspension is sprayed onto 100 g of neutral microspheres (Asahi-Kasei) in a Glatt GPCG1 spray coater.

The granule obtained has a potassium losartan concentration of 81%.

### Example 2

### Preparation of Potassium Losartan Microparticles

200 g of ethylcellulose (Ethocel 20 Premium/Dow), 15 g of Plasdone K29/32® (ISP), 10 g of Cremophor RH 40 (BASF) and 25 g of castor oil are dispersed in a mixture composed of 60% isopropanol and 40% acetone. This solution is sprayed onto 750 g of potassium losartan granules (prepared in example 1).

The microparticles obtained are then placed in a size 2 gelatin gel capsule. The dose of potassium losartan per gel capsule was set in this test at 100 mg (i.e. 165 mg of microparticles). This gel capsule constitutes the final form of the medicament.

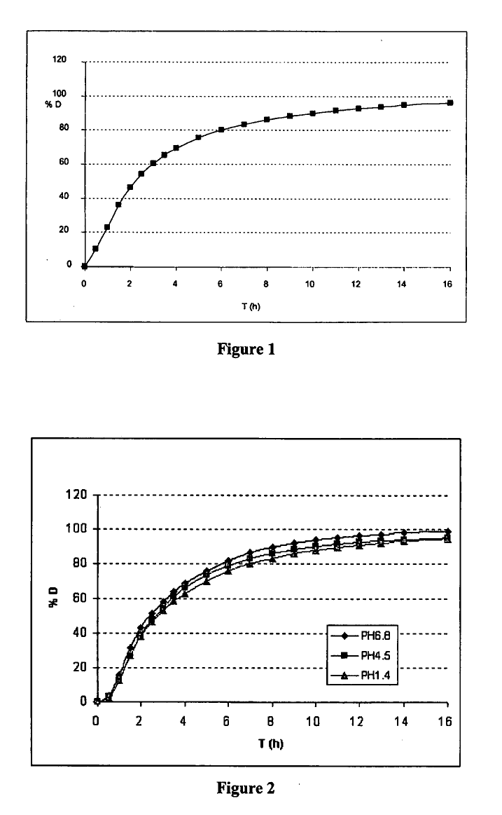

The gel capsule containing the microparticles was tested in a type II dissolutest in accordance with the Pharmacopea at 37° C. and with stirring at 100 rpm at pH 6.8 (0.05 M KH₂PO₄/NaOH). See FIG. 1.

The gel capsule containing the microparticles was tested in a type II dissolutest in accordance with the Pharmacopea at 37° C. and with stirring at 100 rpm at pH 1.4 (HCl); 4.5 (KH₂PO₄/NaOH) and 6.8 (KH₂PO₄/NaOH). See FIG. 2.

It is found that the release of losartan is virtually independent of the pH of the dissolution medium, which makes it possible to have an in vivo release that does not depend on the pH of the gastric juices.

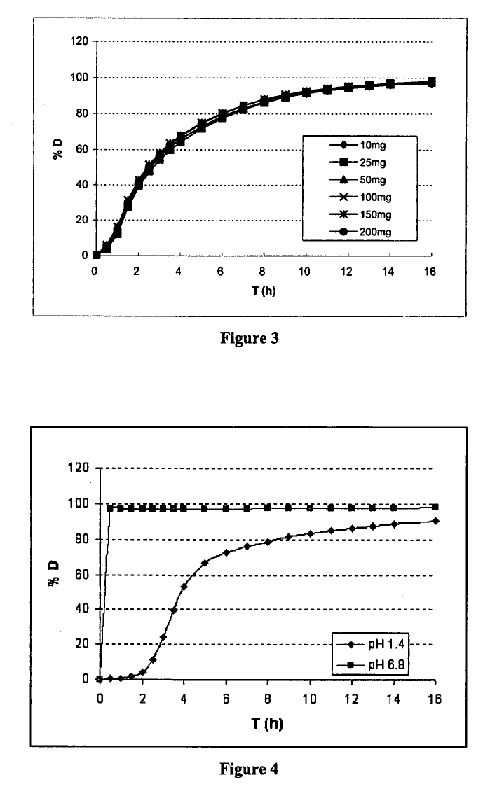

In order to study the influence of the dose of potassium losartan on the dissolution profile, the release of losartan by the microparticles was determined for a dose of 10, 25, 50, 100, 150 and 200 mg of active principle in a type II dissolutest in accordance with the Pharmacopea at 37° C. and with stirring at 100 rpm at pH 6.8 (KH₂PO₄/NaOH). See FIG. 3.

The dissolution profile is independent of the dose of losartan.

### Example 3

### Preparation of Potassium Losartan Microparticles

100 g of hydrogenated cottonseed oil (Penwest) and 150 g of Eudragit® L100-55 (Röhm) are dissolved in hot isopropanol. This solution is sprayed onto 750 g of potassium losartan granules (prepared in example 1).

The microparticles obtained are then placed in a size 2 gelatin gel capsule. The dose of potassium losartan per gel capsule was set in this test at 100 mg (i.e. 165 mg of microparticles). This gel capsule constitutes the final form of the medicament.

The gel capsule containing the microparticles was tested in a type II dissolutest in accordance with the Pharmacopea at 37° C. and with stirring at 100 rpm at pH 1.4 (HCl) and at pH 6.8 (0.05 M KH₂PO₄/NaOH). See FIG. 4.

### Example 4

### Preparation of Potassium Losartan Microparticles

100 g of hydrogenated cottonseed oil (Penwest), 50 g of Eudragit® L100-55 (Röhm) and 100 g of Eudragit® S100 (Röhm) are dissolved in hot isopropanol. The solution is sprayed onto 750 g of potassium losartan granules (prepared in example 1).

The microparticles obtained are then placed in a size 2 gelatin gel capsule. The dose of potassium losartan per gel capsule was set in this test at 100 mg (i.e. 165 mg of microparticles). This gel capsule constitutes the final form of the medicament.

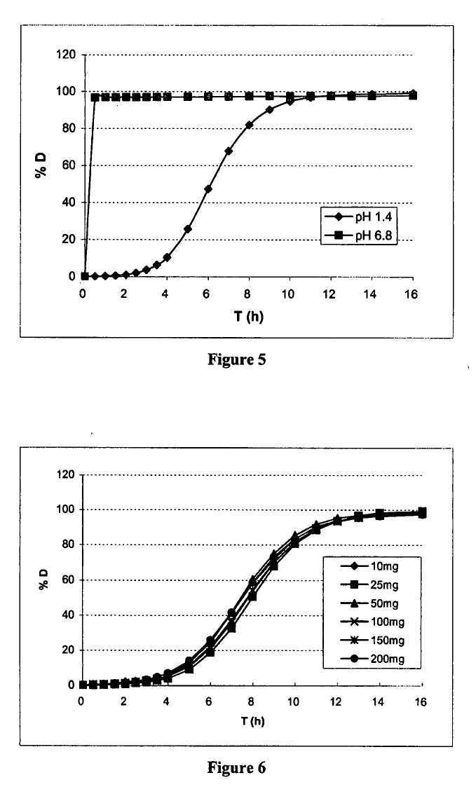

The gel capsule containing the microparticles was tested in a type II dissolutest in accordance with the Pharmacopea at 37° C. and with stirring at 100 rpm at pH 1.4 (HCl) and at pH 6.8 (0.05 M KH₂PO₄/NaOH). See FIG. 5.

In order to study the influence of the dose of potassium losartan on the dissolution profile, the release of losartan by the microparticles was determined for a dose of 10, 25, 50, 100, 150 and 200 mg of active principle in the type II dissolutest test in accordance with the Pharmacopea at 37° C. and with stirring at 100 rpm at pH 1.4 (KH₂PO₄/NaOH). See FIG. 6.

The dissolution profile is independent of the dose of losartan.

### Example 5

### Preparation of Potassium Losartan Microparticles

120 g of ethylcellulose (Ethocel 20 Premium/Dow), 9 g of Plasdone K29/32® (ISP), 6 g of Cremophor RH 40 (BASF) and 15 g of castor oil are dispersed in a mixture composed of 60% isopropanol and 40% acetone. This solution is sprayed onto 850 g of potassium losartan granules (prepared in example 1).

The microparticles obtained are then placed in a size 2 gelatin gel capsule. The dose of potassium losartan per gel capsule was set in this test at 100 mg (i.e. 145 mg of microparticles). This gel capsule constitutes the final form of the medicament.

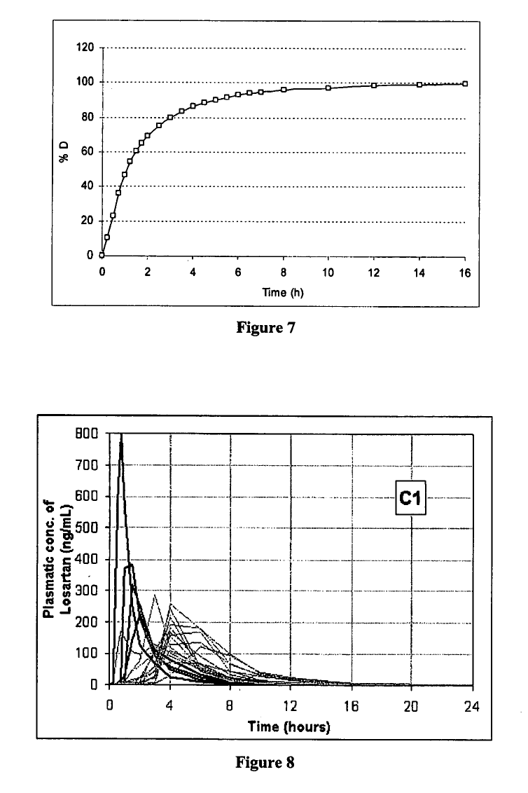

The gel capsule containing the microparticles was tested in a type II dissolutest in accordance with the Pharmacopea at 37° C. and with stirring at 100 rpm at pH 6.8 (0.05 M KH₂PO₄/NaOH). See FIG. 7.

### Example 6

### Tablet Based on Potassium Losartan Microparticles

165 g of the microparticles obtained in example 4, 280 g of lactose, 40 g of crospovidone and 15 g of magnesium stearate are mixed together using an Erweka laboratory mixer.

Tablets containing a 500 mg dose of the above mixture are prepared using a Korsch tablet press. These tablets constitute the final form of the medicament.

The in vitro dissolution profile at pH 1.4 (HCl) of the tablets thus prepared is identical to that of the gel capsules of example 4. See FIG. 5.

It is observed that the release of the potassium losartan is delayed and sustained over a period of about 10 hours, which makes it possible during the administration of such a medicament to increase the bioabsorption times and to comply optimally with the patient's chronobiology.

### Example 7

### In Vivo Data

### Pharmaceutical Forms Used

| | |
|---|---|
| 100 mg Cozaar ® tablet (trade name) | C1 |
| Pharmaceutical form of losartan according to example 2 | M1 |
| Pharmaceutical form of losartan according to example 4 | M2 |

### Description of the Test

The formulations M1 and M2 of examples 2 and 4, and the formulation C1 are administered once a day, and at a dose of 100 mg, after breakfast, to 20 healthy volunteers during a crossed-test study. The plasmatic concentrations of losartan and of EXP3174 (its main active metabolite) are measured at times: 0-0.25-0.5-0.75-1-1.5-2-3-4-6-8-10-12-16-18-20-24-36-48 hours after administration.

### Pharmacokinetic Results

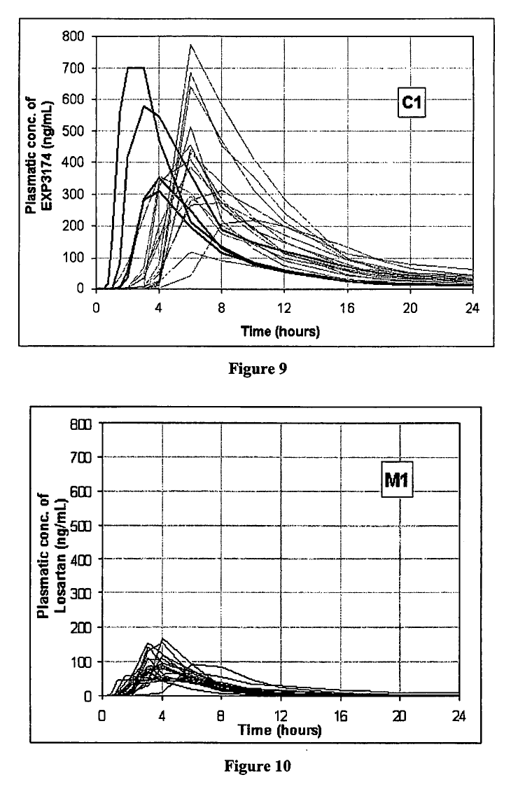

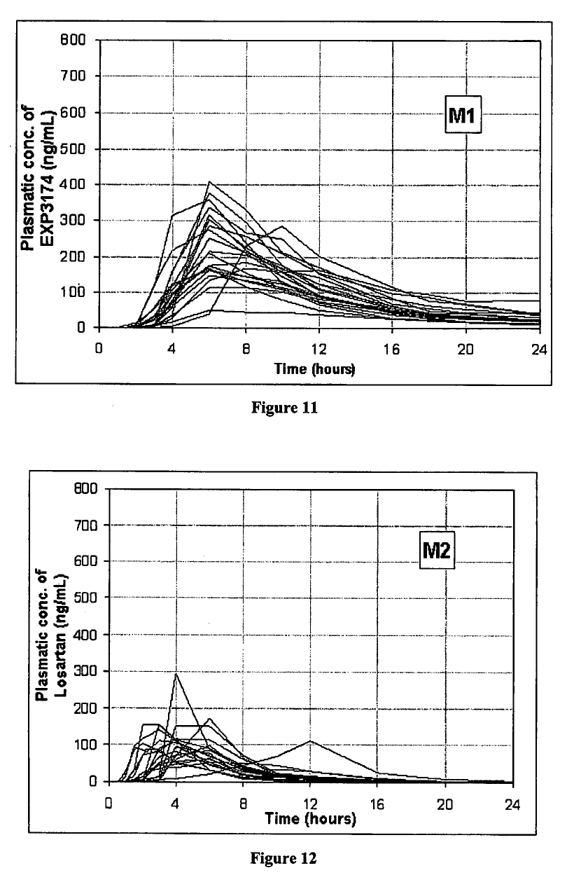

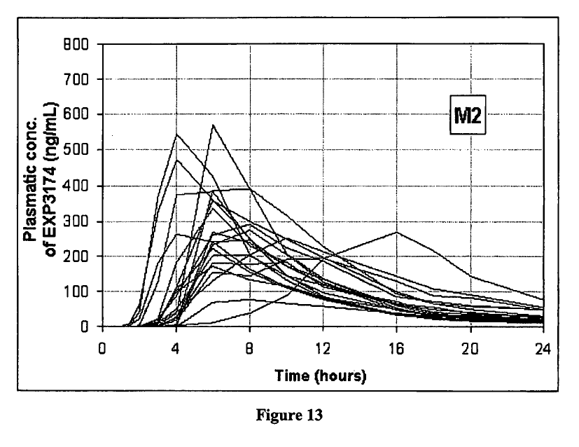

The individual pharmacokinetic profiles of losartan after administration of M1, M2 and C1 are described in FIGS. 10, 12 and 8, respectively. The individual pharmacokinetic profiles of the metabolite EXP3174 after administration of M1, M2 and C1 are described in FIGS. 11, 13 and 9, respectively.

It will be noted that the formulation C1 not included in the context of the invention leads to two populations (rapid Pr and slow Ps) of plasmatic concentration profiles for losartan and for its metabolite EXP3174.

It will also be noted that the formulation C1 leads to a very wide dispersion of the Cmax (high standard deviation).

On the other hand, the formulations M1 and M2 according to the invention lead to only one slow population Ps of profiles and to a low standard deviation of the Cmax values.

The distribution of the number of individuals having, for the losartan profile after administration of C1, M1 and M2, a Tmax of less than 1 and 1.5 hours is given in Table I below:

TABLE 1

Number of individuals (%)

| Treatment | Tmax ≦ 1 h | Tmax ≦ 1.5 h |
|---|---|---|
| C1 | 2 (10%) | 4 (20%) |
| M1 | 0 (0%) | 0 (0%) |
| M2 | 0 (0%) | 1 (5%) |

It is found that the formulations M1 and M2 according to the invention make it possible to reduce considerably the proportion of individuals with a short Tmax relative to the reference form C1.

The mean pharmacokinetic parameters±SD of losartan (Cmax and Tmax) and of its metabolite EXP3174 (Cmax, Tmax, C24h, Cmax/C24h) are given in Table 2 below:

TABLE 2

| | Losartan | | | EXP3174 | | | | |
|---|---|---|---|---|---|---|---|---|
| | Cmax | Tmax | AUC_{0-48 h} | Cmax | Tmax | AUC_{0-48 h} | C24 h | C24 h/ |
| Treatment | (ng/mL) | (h) | ng/(mL × h) | (ng/mL) | (h) | (ng/mL × h) | (ng/mL) | Cmax |
| C1 | 226 ± 159 | 3.5 ± 1.6 | 699 ± 201 | 434 ± 176 | 5.7 ± 1.8 | 3265 ± 1067 | 26 ± 14 | 21 ± 14 |
| M1 | 96 ± 35 | 3.6 ± 0.8 | 481 ± 136 | 235 ± 94 | 6.5 ± 1.1 | 2311 ± 714 | 25 ± 15 | 11 ± 6 |
| M2 | 118 ± 53 | 4.5 ± 2.3 | 559 ± 165 | 298 ± 125 | 7.4 ± 2.9 | 2748 ± 802 | 29 ± 18 | 13 ± 9 |

It is found, relative to the reference form C1 not belonging to the invention, that the formulations M1 and M2 according to the invention make it possible

a) to reduce the standard deviation of the Cmax values

b) to reduce the mean peak/trough modulation

c) to reduce the standard deviation of the peak/trough modulation.

1. The use, in an oral pharmaceutical form comprising losartan, of a coating or matrix including said losartan and allowing controlled release of said losartan, such that this form orally administered to a sample of individuals leads, irrespective of the fed or fasted state of the individuals, to a reduction of the interindividual standard deviation of the Cmax, which ensures lower variability of the efficacy and of the therapeutic safety of the pharmaceutical form relative to an immediate-release pharmaceutical form of losartan administered to this same sample of individuals, at the same dose.

2. The use of losartan contained in a coating or matrix that allows controlled release of said losartan, for manufacturing an oral pharmaceutical form which, after oral administration to a sample of individuals, leads, irrespective of the fed or fasted state of the individuals, to a reduction of the interindividual standard deviation of the Cmax, which ensures lower variability of the efficacy and of the therapeutic safety of the pharmaceutical form relative to an immediate-release pharmaceutical form of losartan administered to this same sample of individuals, at the same dose.

3. The use as claimed in claim 1 or 2, characterized in that the factor (f) for reduction of the interindividual standard deviation of the Cmax is defined as follows: f≧1.2; preferably f≧1.75, and even more preferentially f is between 2.5 and 20.

4. The use as claimed in at least one of the preceding claims, characterized in that the coating or matrix of the pharmaceutical form is designed such that it allows the controlled release of losartan, firstly to avoid any premature and/or massive and/or rapid release of losartan and subsequently any deleterious plasmatic overconcentration of losartan, and secondly to ensure therapeutic cover between two dose intakes.

5. The use as claimed in at least one of the preceding claims, characterized in that the coating or matrix of the pharmaceutical form is designed such that the oral administration of this form to a sample of individuals leads to a mean peak/trough modulation of the plasmatic profiles of the metabolite EXP3174 that is less than the mean peak/trough modulation of the metabolite EXP3174 for the same sample of individuals receiving the same dose of an immediate-release form of losartan.

6. The use as claimed in at least one of the preceding claims, characterized in that the coating or matrix of the pharmaceutical form is designed such that the oral administration of this form to a sample of individuals leads to a variability of the peak/trough modulation of the plasmatic profiles for the metabolite EXP3174 that is less than the variability of the peak/trough modulation of the metabolite EXP3174 for the same sample of individuals receiving the same dose of an immediate-release form of losartan.

7. The use as claimed in at least one of the preceding claims, characterized in that the oral pharmaceutical form contains losartan in the form of microunits, which may be:
microparticles individually consisting of a core that comprises losartan and that is coated with at least one coating allowing the controlled release of losartan;
and/or microgranules individually consisting of a matrix that includes losartan and that allows the controlled release of losartan;
and/or immediate-release losartan microgranules.

8. The use as claimed in at least one of claims 1 to 6, characterized in that the oral pharmaceutical form is a tablet free of microparticles individually consisting of a core comprising losartan and coated with at least one coating allowing the controlled release of losartan and/or free of microgranules individually consisting of a matrix including losartan and allowing the controlled release of losartan.

9. The use as claimed in at least one of the preceding claims, characterized in that the pharmaceutical form makes it possible to obtain, after a dose intake, a plasmatic profile defined as follows:

| | Cmax/C24 h ≦ Cmax*/C24 h* |
|---|---|
| preferably | 1.5 × Cmax/C24 h ≦ Cmax*/C24 h* |
| and even more preferentially | 2.0 × Cmax/C24 h ≦ Cmax*/C24 h* |

with:
C24h representing the mean plasmatic concentration of the active metabolite EXP3174 of losartan, 24 hours after the dose intake,
C24h* representing the mean plasmatic concentration of EXP3174 obtained under the same conditions as C24h, with a reference immediate-release oral pharmaceutical form, containing the same dose of losartan,
Cmax representing the mean maximum plasmatic concentration of EXP3174 after the dose intake,
Cmax* representing the mean maximum plasmatic concentration of EXP3174 obtained under the same conditions as Cmax, with a reference immediate-release oral pharmaceutical form containing the same dose of losartan.

10. The use as claimed in claim 7, characterized in that the oral pharmaceutical form comprises microparticles and has an in vitro dissolution profile [D % (t)] such that the time t (70%) after the administration and at the end of which 70% of the losartan is released is between 1 and 24 hours, preferably between 2 and 12 hours and even more preferentially between 2 and 8 hours.

11. The use as claimed in claim 10, characterized in that the in vitro dissolution profile [D % (t)] of the oral pharmaceutical form is such that, for any value of the time t of between 2 hours and t(70%), preferably for any value of the time t of between 1 hour and t(70%), the percentage of dissolved (released) losartan [D % (t)]≧35×t/t(70%).

12. The use as claimed in at least one of the preceding claims, characterized in that the rate of release of losartan in an in vitro dissolution test is independent of the pH.

13. The use as claimed in claim 7, characterized in that the oral pharmaceutical form is such that:
the release of losartan is governed by two separate initiating mechanisms, one being based on a pH variation and the other allowing the release of the losartan, after a predetermined residence time in the stomach;
at a constant pH of 1.4, the dissolution profile comprises a lag phase with a duration of less than or equal to 7 hours, preferably less than or equal to 5 hours and even more preferentially between 1 and 5 hours,
and passing from pH 1.4 to pH 7.0 leads to a release phase starting without a lag time.

14. The use as claimed in claim 13, characterized in that the oral pharmaceutical form has a dissolution profile, measured in an in vitro dissolution test, as indicated below:
less than 20% of the losartan is released after 2 hours at pH 1.4;
at least 50% of the losartan is released after 16 hours at pH 1.4.

15. The use as claimed in claim 13, characterized in that the oral pharmaceutical form comprises controlled-release losartan microparticles whose initiating pH is between 6.0 inclusive and 6.5 inclusive.

16. The use as claimed in one of claims 7 and 10 to 15, characterized in that the oral pharmaceutical form comprises at least two populations of microparticles.

17. The use as claimed in one of claims 7 and 10 to 16, characterized in that the oral pharmaceutical form comprises at least one population of controlled-release microparticles and/or microgranules and/or at least one population of immediate-release microgranules.

18. The use as claimed in one of claims 7 and 13 to 17, characterized in that the oral pharmaceutical form comprises at least two populations of controlled-release microparticles and/or microgranules with different dissolution profiles, for at least one pH value of between 1.4 and 7.4.

19. The use as claimed in one of claims 7 and 13 to 18, characterized in that the oral pharmaceutical form comprises at least two populations of controlled-release microparticles and/or microgranules that differ in their respective initiating pHs.

20. The use as claimed in one of claims 7 and 13 to 19, characterized in that the oral pharmaceutical form comprises at least two populations of controlled-release losartan microparticles and/or microgranules that differ in their respective initiating times.

21. The use as claimed in one of claims 7 and 13 to 20, characterized in that the oral pharmaceutical form comprises:
at least one population of immediate-release losartan microgranules;
at least one population P1 of controlled-release losartan microparticles and/or microgranules, and
at least one population P2 of controlled-release losartan microparticles and/or microgranules;
and in that the respective initiating pHs of P1 and of P2 differ by at least 0.5 pH unit, preferably by at least 0.8 pH unit and even more preferentially by at least 0.9 pH unit.

22. The use as claimed in one of claims 7 and 13 to 21, characterized in that the respective initiating pHs of the various populations of controlled-release losartan microparticles and/or microgranules are between 5 and 7.

23. The use as claimed in one of claims 7 and 13 to 22, characterized in that the oral pharmaceutical form comprises:
at least one population of immediate-release losartan microgranules;
at least one population P1′ of controlled-release losartan microparticles and/or microgranules whose initiating pH is equal to 5.5; and
at least one population P2′ of controlled-release losartan microparticles and/or microgranules whose initiating pH is between 6.0 inclusive and 6.5 inclusive.

24. The use as claimed in one of claims 7 to 23, characterized in that the oral pharmaceutical form comprises at least one population of immediate-release losartan microgranules whose behavior in an in vitro dissolution test is such that at least 80% of the losartan is released in 1 hour at any pH of between 1.4 and 7.4.

25. The use as claimed in one of claims 7 to 24, characterized in that the oral pharmaceutical form is in the form of a single daily oral dose comprising from 1000 to 500 000 microunits containing losartan.

26. The use as claimed in one of claims 7 to 25, characterized in that the oral pharmaceutical form is in the form of a single daily oral dose comprising from 1000 to 500 000 controlled-release losartan microparticles and/or microgranules.

27. The use as claimed in one of the preceding claims, characterized in that the oral pharmaceutical form is in the form of a sachet of powder, a liquid suspension, a tablet or a gel capsule.

28. The use as claimed in one of the preceding claims, characterized in that the pharmaceutical form comprises at least one active principle AP other than losartan.

29. The use as claimed in at least one of claims 7 and 10 to 12, characterized in that the pharmaceutical form comprises controlled-release losartan microparticles and/or microgranules for which the composition of the coating or matrix is chosen from the group comprising formula A and formula B described below:
Formula A
A-1—at least one film-forming polymer (P1) that is insoluble in the fluids of the tract, present in a proportion of from 50% to 90% and preferably 50% to 80% by weight of solids relative to the total mass of the coating composition and especially comprising at least one water-insoluble cellulose derivative;
A-2—at least one nitrogenous polymer (P2) present in a proportion of from 2% to 25% and preferably 5% to 15% by weight of solids relative to the total mass of the coating composition and consisting of at least one polyacrylamide and/or one poly-N-vinylamide and/or one poly-N-vinyllactam;
A-3—at least one plasticizer present in a proportion of from 2% to 20% and preferably from 4% to 15% by weight of solids relative to the total mass of the coating composition and consisting of at least one of the following compounds: glycerol esters, phthalates, citrates, sebacates, cetyl alcohol esters, castor oil;
A-4—at least one surfactant and/or lubricant, present in a proportion of from 2% to 20% and preferably from 4% to 15% by weight of solids relative to the total mass of the coating composition and chosen from anionic surfactants and/or from nonionic surfactants and/or from lubricants; said agent possibly comprising only one or a mixture of the abovementioned products;
or
Formula B
B1—at least one film-forming polymer that is insoluble in the fluids of the gastrointestinal tract,
B2—at least one water-soluble polymer,
B3—at least one plasticizer,
B4—and optionally at least one surfactant/lubricant preferably consisting of at least one anionic surfactant and/or at least one nonionic surfactant.

30. The use as claimed in at least one of claims 7 to 29, characterized in that the controlled-release losartan microparticles and/or microgranules have a mean diameter (Dm in μm) of less than 1000, preferably between 50 and 800 and even more preferentially between 50 and 500.

31. A modified-release oral pharmaceutical form of losartan, characterized
in that it comprises a plurality of microunits containing losartan,
in that the mean diameter (Dm in μm) of the microunits is between 50 and 1000, preferably between 100 and 600 and even more preferentially between 150 and 500,
and in that it makes it possible to obtain, after a dose intake, a plasmatic profile defined as follows:

| | C18 h* ≦ C18 h |
|---|---|
| preferably | 1.5 × C18 h* ≦ C18 h ≦ Cmax*/2 |
| and even more preferentially | 2.0 × C18 h* ≦ C18 h ≦ Cmax*/2 |

with:
C18h representing the plasmatic concentration of the active metabolite (E3174) of losartan, 18 hours after the dose intake,
C18h* representing the plasmatic concentration of E3174 obtained under the same conditions as C18h, with a reference immediate-release oral pharmaceutical form containing the same dose of losartan,
Cmax representing the maximum plasmatic concentration of E3174 after the dose intake,
Cmax* representing the maximum plasmatic concentration of E3174 obtained under the same conditions as Cmax, with a reference immediate-release oral pharmaceutical form containing the same dose of losartan.

32. A modified-release oral pharmaceutical form of losartan, characterized
in that it comprises a plurality of microunits containing losartan,
in that the mean diameter (Dm in μm) of the microunits is between 50 and 1000, preferably 100 and 600 and even more preferentially between 150 and 500,
and in that it makes it possible to obtain, after a dose intake, a plasmatic profile defined as follows:

| -a- | C18 h* ≦ C18 h |
|---|---|
| preferably | 1.5 × C18 h* ≦ C18 h ≦ Cmax*/2 |
| and even more preferentially | 2.0 × C18 h* ≦ C18 h ≦ Cmax*/2 |
| -b- | 1.1 × Tmax* ≦ Tmax |
| preferably | 1.2 × Tmax* ≦ Tmax |
| more preferentially | 1.5 × Tmax* ≦ Tmax |
| and even more preferentially | 1.7 × Tmax* ≦ Tmax ≦ 6 × Tmax |

with:
C18h representing the plasmatic concentration of active metabolite (E3174) of losartan, 18 hours after the dose intake,
C18h* representing the plasmatic concentration of E3174 obtained under the same conditions as C18h, with a reference immediate-release oral pharmaceutical form containing the same dose of losartan,
Cmax representing the maximum plasmatic concentration of E3174 after the dose intake,
Tmax representing the time elapsed after the dose intake and which corresponds to Cmax,
Cmax* representing the maximum plasmatic concentration of E3174 obtained under the same conditions as Cmax, with a reference immediate-release oral pharmaceutical form containing the same dose of losartan,
Tmax* representing the time elapsed after the dose intake and which corresponds to Cmax*.

33. The oral pharmaceutical form as claimed in claim 31 or 32, characterized in that at least some of the microunits are microparticles individually consisting of a core that may comprise losartan and that is coated with at least one coating allowing the modified release of the losartan.

34. The oral pharmaceutical form as claimed in any one of claims 31 to 33, characterized in that at least some of the microunits it comprises consist of immediate-release losartan microgranules.

35. The oral pharmaceutical form as claimed in claim 34, characterized by an in vitro dissolution profile such that: 70% of the losartan is released between 1 and 24 hours, preferably between 2 and 12 hours and even more preferentially between 2 and 8 hours after the administration.

36. The oral pharmaceutical form as claimed in one of claims 31 or 32 and 33, characterized in that:
the release of losartan is governed by two separate initiating mechanisms, one being based on a pH variation and the other allowing the release of the losartan, after a predetermined residence time in the stomach;
at a constant pH of 1.4, the dissolution profile comprises a lag phase with a duration of less than or equal to 7 hours, preferably less than or equal to 5 hours and even more preferentially between 1 and 5 hours,
and passing from pH 1.4 to pH 7.0 leads to a release phase starting without a lag time.

37. The oral pharmaceutical form as claimed in claim 36 and optionally one of claims 40 to 46, characterized in that its dissolution profile, measured in an in vitro dissolution test, is as indicated below:
less than 20% of the losartan is released after 2 hours at pH 1.4;
at least 50% of the losartan is released after 16 hours at pH 1.4.

38. The oral pharmaceutical form as claimed in any one of claims 31 to 37, characterized in that the variability CV (in %) of the area under the curve (AUC) of the plasmatic concentration of active metabolite E3174, as a function of the time (T) after the dose intake, is less than or equal to 200%, preferably 150% and even more preferentially 120% of the corresponding variability CV* (in %) of the area under the curve (AUC*) of the plasmatic concentration of active metabolite E3174, as a function of the time (T) after the dose intake under the same conditions, of a reference immediate-release oral pharmaceutical form*, containing the same dose of losartan, i.e.: CV≦2.0×CV*, CV≦1.5×CV* and preferably CV≦1.2×CV*.

39. The oral pharmaceutical form as claimed in one of claims 31, 33 or 35, characterized in that the in vitro rate of release of losartan in a dissolution test is independent of the pHs.

40. The oral pharmaceutical form as claimed in claim 39, characterized in that the dissolution profiles of the microparticles between pH 1 and pH 5 are similar.

41. The oral pharmaceutical form as claimed in any one of claims 31 to 40, characterized in that it comprises at least two populations of microparticles as claimed in claim 33.

42. The oral pharmaceutical form as claimed in any one of claims 31 to 41, characterized in that it comprises at least one population of microparticles as claimed in claim 33 and at least one population of microgranules as claimed in claim 34.

43. The oral pharmaceutical form as claimed in claim 36 and optionally claim 41, characterized in that it comprises at least two populations of microparticles with different dissolution profiles, for at least one pH value of between 1.4 and 7.4.

44. The oral pharmaceutical form as claimed in claim 36 and optionally one of claims 41 and 43, characterized in that it comprises at least two populations of modified-release losartan microparticles that differ in their respective initiating pH.

45. The oral pharmaceutical form as claimed in claim 36 and optionally one of claims 41, 43 and 44, characterized in that it comprises at least two populations of microparticles with modified release of active principle that differ in their respective initiating times.

46. The oral pharmaceutical form as claimed in claim 36 and optionally one of claims 41 and 43 to 45, characterized in that it comprises:
at least one population of immediate-release losartan microgranules;
at least one population P₁ of modified-release losartan microparticles, and
at least one population P₂ of modified-release losartan microparticles;
and in that the respective initiating pHs of P₁ and of P₂ differ by at least 0.5 pH unit, preferably by at least 0.8 pH unit and even more preferentially by at least 0.9 pH unit.

47. The oral pharmaceutical form as claimed in claim 36 and optionally one of claims 41 and 43 to 46, characterized in that the respective initiating pHs of the various populations of modified-release losartan microparticles are between 5 and 7.

48. The oral pharmaceutical form as claimed in claim 36 and optionally one of claims 41 to 47, characterized in that it comprises:
at least one population of immediate-release losartan microgranules;
at least one population P₁′ of modified-release losartan microparticles whose initiating pH is equal to 5.5; and
at least one population P₂′ of modified-release losartan microparticles whose initiating pH is between 6.0 inclusive and 6.5 inclusive.

49. The oral pharmaceutical form as claimed in any one of claims 34 to 48, characterized in that it comprises at least one population of immediate-release losartan microgranules whose behavior in an in vitro dissolution test is such that at least 80% of the losartan is released in 1 hour at any pH of between 1.4 and 7.4.

50. The oral pharmaceutical form as claimed in any one of claims 31 to 49, characterized in that at least 50% of the losartan is in its crystalline form I.

51. The oral pharmaceutical form as claimed in one of claims 33 to 50, characterized in that at least some of the modified-release losartan microparticles each comprise:
a core containing losartan and
at least one coating covering the core and allowing the modified release of said losartan.

52. The oral pharmaceutical form as claimed in any one of claims 32 to 50, characterized in that at least some of said modified-release losartan microparticles each comprise:
a core comprising:
a neutral core,
at least one active layer comprising the losartan and coating the neutral core,
and at least one coating covering the core and allowing the modified release of the losartan.

53. The oral pharmaceutical form as claimed in any one of the preceding claims, characterized in that the proportion of losartan in the microunits (expressed as a weight percentage of solids relative to the total mass of the microunits) is between 5 and 80, preferably between 10 and 70 and even more preferentially between 15 and 60.

54. The oral pharmaceutical form as claimed in claim 33 and optionally any one of claims 34 to 52, characterized in that the immediate-release losartan microgranules are uncoated microparticle cores as claimed in claim 34.

55. The oral pharmaceutical form as claimed in any one of the preceding claims, characterized in that it is in the form of a single daily oral dose comprising from 1000 to 500 000 microunits containing losartan.

56. The oral pharmaceutical form as claimed in any one of the preceding claims, characterized in that it is in the form of a single daily oral dose comprising from 1000 to 500 000 modified-release losartan microparticles.

57. The oral pharmaceutical form as claimed in any one of claims 31 to 56, characterized in that it is in the form of a sachet of powder of microunits, a liquid suspension of microparticles, a tablet obtained from microunits, or a gel capsule containing microunits.

58. The use of the modified-release losartan microparticles as defined in any one of claims 31 to 57 and optionally of the immediate-release losartan microgranules as defined in any one of claims 34 to 57, for the preparation of pharmaceutical or dietetic microparticulate oral galenical forms, preferably in the form of tablets that are advantageously orodispersible, or powders or gel capsules.

59. The use of the modified-release losartan microparticles as defined in any one of claims 31 to 58 and optionally of the immediate-release losartan microgranules as defined in any one of claims 34 to 58, for the preparation of a therapeutically safe microparticulate oral pharmaceutical form, designed such that once said pharmaceutical form has been ingested, the microparticles it comprises are dispersed and individualized when they reach the stomach, which allows these microparticles to undergo uniform and gradual gastric emptying, whether the patient is in the fed or fasted state during the dose intake, thus ensuring release of the losartan in its gastrointestinal bioabsorption window.

60. The microparticles as defined in any one of the preceding claims.
